(19) United States
(12) Patent Application Publication
(10) Pub. No.: US 2024/0304303 A1
(43) Pub. Date: Sep. 12, 2024

(54) METHOD FOR DETERMINING A COMPLEMENTARY FEEDING DIET

(71) Applicant: SOCIETE DES PRODUITS NESTLE S.A., Vevey (CH)

(72) Inventors: LYNDA O'NEILL, Chatel-St-Denis (CH); ANNE DATTILO, Hollywood, FL (US); JOSE M. SAAVEDRA, Morristown, NJ (US); MATTHIEU MAILLOT, Marseille (FR); FLORENT VIEUX, Marseille (FR)

(21) Appl. No.: 18/551,915

(22) PCT Filed: Mar. 24, 2022
(86) PCT No.: PCT/EP2022/057810

§ 371 Date: Sep. 22, 2023

(30) Foreign Application Priority Data
Mar. 24, 2021 (EP) 21164672.4

(51) Int. Cl. G16H 20/60; A23L 33/00

(52) U.S. Cl. CPC G16H 20/60; A23L 33/30; A23L 33/40

(57) ABSTRACT

The present invention provides a method for determining a complementary feeding diet providing a plurality of food types to meet the nutrient and energy requirements of a child's diet during the complementary feeding period, comprising: (a) providing (i) nutrient targets for a plurality of nutrients and (ii) average intakes for the plurality of food types; and (b) determining the complementary feeding diet by minimising the weighted sum of (i) the average negative deviation between the nutrient targets and the amount of each nutrient in the child's diet and (ii) the average deviation between the average intakes and the amount of each food type in the child's diet.

### FIELD OF THE INVENTION

The present invention relates to methods for determining a complementary feeding diet, and/or fortificant and/or supplement doses to meet the requirements of a child's diet during the complementary feeding period. The present invention also relates to complementary feeding diets, fortificants and supplement doses obtained by said methods and uses thereof.

### BACKGROUND TO THE INVENTION

Infants and toddlers in most regions have suboptimal diets (Denney L, et al. J Nutr Health Food Sci. 2016; 4(6):1-8). Global Feeding Infants and Toddlers Studies (FITS) have indicated that diets are too low in fruits and vegetables, dietary diversity is low, and energy intakes are in excess of requirements (Dwyer J T. The Journal of Nutrition. 2018; 148(suppl 3):1575S-80S). Studies also demonstrate that long-term dietary patterns are established by the age of 24 months (Nicklaus S. Ann Nutr Metab. 2017; 70(3):241-5). Therefore, the diet consumed during the complementary feeding period is critically important for laying the foundation for long-term healthy eating habits (Luque V, et al. The Journal of Nutrition. 2018; 148(5):752-9).

In many countries, nutrient intakes are suboptimal, with inadequate intakes of iron, calcium, and zinc observed among infants and toddlers. Total fat and fiber intakes are frequently too low and sodium intakes are excessive among toddlers. In addition, excess protein intakes are prevalent among infants (Fewtrell M, et al. Journal of pediatric gastroenterology and nutrition. 2017; 64(1):119-32).

While nutrient recommendations are published by global, regional and/or country specific authoritative organisations, there are limited quantitative food based dietary guidelines (FBDG) to meet them during the complementary feeding period. Only five countries have created quantitative FBDG for the complementary feeding period and when these are translated into weekly menu plans to determine their adequacy relative to local and World Health Organisation (WHO) nutrient recommendations, they all fall short for some micronutrients, particularly for breast-fed infants. Moreover, all diets provide high protein relative to the European Society for Pediatric Gastroenterology Hepatology and Nutrition (ESPGHAN) recommendation that protein should be <15% of total energy. Diets for formula-fed infants also tend to exceed the zinc upper level (UL) recommended by the Institute of Medicine (IOM).

FBDG have mainly been created in low- and middle-income countries and are lacking for infants and toddlers in developed countries according to age (e.g. disaggregating 6-12 month olds into two smaller age segments), breastfeeding status, and with consideration of different fortification scenarios.

Thus, systematic methods for determining quantitative feeding recommendations during the complementary feeding period are required.

### SUMMARY OF THE INVENTION

The present inventors have found that mathematical programming can be used to generate optimised complementary food diets for infants and toddlers in developed countries according to age, breast feeding status, and fortification of the food supply. Further, mathematical programming can be used to identify nutrient requirements that are difficult to meet or cannot be met by complementary food diets and this can be used to determine appropriate fortificant and/or supplement doses for said infants and toddlers.

In one aspect, the present invention provides a method for determining a complementary feeding diet providing a plurality of food types to meet the nutrient and energy requirements of a child's diet during the complementary feeding period. In some embodiments, the method comprises:
(a) providing (i) nutrient targets for a plurality of nutrients and (ii) average intakes for the plurality of food types; and
(b) determining the complementary feeding diet by minimising the weighted sum of (i) the average negative deviation between the nutrient targets and the amount of each nutrient in the child's diet and (ii) the average deviation between the average intakes and the amount of each food type in the child's diet.

In one aspect, the present invention provides a method for determining fortificant and/or supplement doses to meet the nutrient requirements of a child's diet during the complementary feeding period. In some embodiments, the method comprises:
(a) providing (i) nutrient targets for a plurality of nutrients and (ii) average intakes for the plurality of food types;
(b) determining a complementary feeding diet providing a plurality of food types by minimising the weighted sum of (i) the average negative deviation between the nutrient targets and the amount of each nutrient in the child's diet and (ii) the average deviation between the average intakes and the amount of each food type in the child's diet; and
(c) determining fortificant and/or supplement doses required to meet the nutrient requirements of the child's diet during the complementary feeding period.

Typically, the complementary feeding period is when the child is aged from 6 months to less than 24 months. In some embodiments, the child is aged from 6 months to less than 9 months, from 9 months to less than 12 months, or from 12 months to less than 24 months.

Typically, the child's diet comprises the complementary feeding diet and non-complementary food. Optionally, the child's diet further comprises one or more supplements. In some embodiments, the child's diet comprises the complementary feeding diet and one or more of breast milk, infant formula, and cow's milk. In some embodiments, the child's diet comprises the complementary feeding diet and breast milk; the complementary feeding diet and infant formula; the complementary feeding diet, breast milk and infant formula; or the complementary feeding diet and cow's milk. In some embodiments:
(i) the child is aged from 6 months to less than 9 months and the child's diet comprises the complementary feeding diet and breast milk;
(ii) the child is aged from 6 months to less than 9 months and the child's diet comprises the complementary feeding diet and infant formula;
(iii) the child is aged from 6 months to less than 9 months and the child's diet comprises the complementary feeding diet, breast milk and infant formula;
(iv) the child is aged from 9 months to less than 12 months and the child's diet comprises the complementary feeding diet and breast milk;
(v) the child is aged from 9 months to less than 12 months and the child's diet comprises the complementary feeding diet and infant formula;
(vi) the child is aged from 9 months to less than 12 months and the child's diet comprises the complementary feeding diet, breast milk and infant formula; or
(vii) the child is aged from 12 months to less than 24 months and the child's diet comprises the complementary feeding diet and cow's milk.

The nutrient and energy requirements of the child may be determined based on published guidelines. In some embodiments, the energy requirements of a child's diet are determined based on the weight of the child, preferably wherein the weight of the infant is determined based on body weight values from 50th percentile of the Growth Chart Percentiles from the World Health Organization (WHO). In some embodiments, the child's diet is isocaloric, optionally wherein:
(i) the child is aged from 6 months to less than 9 months and the child's diet comprises about 636 kcal/day;
(ii) the child is aged from 9 to less than 12 months and the child's diet comprises about 706 kcal/day; or
(iii) the child is aged from 12 months to less than 24 months and the child's diet comprises about 860 kcal/day.

The complementary feeding diet may be fortified or unfortified. Suitably, the complementary feeding diet is diverse, age-appropriate, palatable, realistic, and generalizable, or any combination thereof. In preferred embodiments, the complementary feeding diet is diverse.

In some embodiments, the complementary feeding diet meets at least the following criteria: (a) at least 10 g/day of two types of fruit; (b) at least 10 g/day of three types of vegetable; (c) no more than 50 g/day egg and no more than 150 g/week egg; (d) no more than 21 g/day of any one type of meat; and (e) optionally, a limit of 10% total energy from occasional foods.

In some embodiments, the plurality of food types comprises at least: one or more types of fruit; one or more types of vegetable; one or more types of pulse, nut or seed; one or more types of meat; one or more types of starch or grain; one or more type of dairy; and one or more type of oil and fat. In some embodiments, the plurality of food types excludes one or more food types, optionally wherein the one or more excluded food types comprise one or more of: (a) foods with low or no nutrient contribution; (b) developmentally or age inappropriate foods; (c) occasional foods, for example 100% fruit juice; (d) supplements; (e) foods for special diets; (f) regional-specific foods; and (g) mixed foods.

The optimised complementary feeding diet may be determined by any mathematical optimisation or mathematical programming method known in the art. In some embodiments, the complementary feeding diet is determined using linear programming.

Nutrient targets can be provided for any nutrient and may be determined based on published guidelines. In some embodiments, the nutrient targets comprise targets for at least protein, lipids, iron, calcium, and zinc. In preferred embodiments, the nutrient targets further comprise targets for ALA and/or DHA. In preferred embodiments, the nutrient targets further comprise targets for one or more of magnesium, phosphorus, sodium, and potassium. In preferred embodiments, the nutrient targets further comprise targets for vitamin A, vitamin C, and folate. In preferred embodiments, the nutrient targets further comprise targets for one or more of vitamin D, vitamin B6, vitamin B12, vitamin E. In some embodiments, the nutrient targets take into account bioavailability for one or more nutrient, preferably the target for iron takes into account bioavailability of iron.

Any suitable weighting may be applied to the weighted sum. In some embodiments, the weighted sum gives a greater weighting to (i) the average negative deviation between the nutrient targets and the amount of each nutrient in the child's diet.

In another aspect, the present invention provides a computer program comprising instructions which, when the program is executed by a computer, cause the computer to carry out the method of the present invention.

In another aspect, the present invention provides a computer-readable data carrier having stored thereon the computer program of the present invention.

In another aspect, the present invention provides a computer-readable medium comprising instructions which, when executed by a computer, cause the computer to carry out the method of the present invention.

In another aspect, the present invention provides complementary feeding diet obtained by the method of the present invention and use of said complementary feeding diet to meet the nutrient and energy requirements of a child during the complementary feeding period.

In another aspect, the present invention provides fortificant and/or supplement doses obtained by the method of the present invention and use of said fortificant and/or supplement doses to supplement a child's diet during the complementary feeding period. In some embodiments:
(a) the fortificant and/or supplement dose is a vitamin D fortificant and/or supplement dose and the child is aged from 6 months to less than 9 months, from 9 months to less than 12 months, or from 12 months to less than 24 months;
(b) the fortificant and/or supplement dose is a DHA fortificant and/or supplement dose and the child is aged from 12 months to less than 24 months; or
(c) the fortificant and/or supplement dose is for an iron fortificant and/or supplement dose and the child is from 6 months to less than 9 months and is breast-fed.

### DESCRIPTION OF DRAWINGS

FIG. 1—Exemplary infant populations according to age, breast feeding status and fortification of the food supply

FIG. 2—Exemplary energy from milk versus complementary foods by age

The ratio of milk to complementary food in the diet is based on published observations. The volume of milk is calculated based on the proportion of energy from milk versus complementary food and the estimated energy requirements (EER) per day according to age.

FIG. 3—Exemplary major and minor food groups

7 exemplary major food groups and 38 exemplary food subgroups. The numbers in parentheses represent the number of individual foods per minor group by age group. Items in bold were not introduced in the model until the 9-11.9 month old infant diets. Items with an asterisk were not introduced until 12-23.9 month old toddler diets

FIG. 4—Exemplary energy, macro and micronutrient targets, minima, and maxima per age

The macronutrient targets are mainly from WHO/FAO with the exception of the maximum limit for protein, which is based on ESPGHAN recommendations. The micronutrient targets are mainly from WHO/FAO with the exception of sodium, potassium, magnesium, vitamin B12, vitamin D and dietary fiber, which are based on other published recommendations.

FIG. 5—Exemplary optimised diets: 6 to 8.9 month old infants excluding milk

Exemplary optimised diets for 6 to 8.9 month old infants excluding milk, based on (A) g/day and (B) kcal/day. Table (C) shows exemplary major food group intakes in g/day in observed and optimised diets. Table (D) shows exemplary nutrient requirements and targets and the nutrients provided by the exemplary optimised diets. The infants are those identified as sub-population SP1 shown in FIG. 1.

FIG. 6—Exemplary optimised diets: 9 to 11.9 month old infants excluding milk

Exemplary optimised diets for 9 to 11.9 month old infants excluding milk, based on (A) g/day and (B) kcal/day. Table (C) shows exemplary major food group intakes in g/day in observed and optimised diets. Table (D) shows exemplary nutrient requirements and targets and the nutrients provided by the exemplary optimised diets. The infants are those identified as sub-population SP2 shown in FIG. 1.

FIG. 7—Exemplary optimised diets: 12 to 23.9 month old toddlers excluding milk

Exemplary optimised diets for 12 to 23.9 month old toddlers excluding milk, based on (A) g/day and (B) kcal/day. Table (C) shows exemplary major food group intakes in g/day in observed and optimised diets. Table (D) shows exemplary nutrient requirements and targets and the nutrients provided by the exemplary optimised diets. The infants are those identified as sub-population SP3 shown in FIG. 1.

FIG. 8—Example of a daily menu for a breast-fed 6 to 8.9 months old on a fortified diet

### DETAILED DESCRIPTION

Various preferred features and embodiments of the present invention will now be described by way of non-limiting examples. The skilled person will understand that they can combine all features of the invention disclosed herein without departing from the scope of the invention as disclosed.

As used herein, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

Numeric ranges are inclusive of the numbers defining the range unless otherwise indicated.

The publications discussed herein are provided solely for their disclosure prior to the filing date of the present application. Nothing herein is to be construed as an admission that such publications constitute prior art to the claims appended hereto.

### Complementary Feeding Diet

The present invention provides a method for determining a complementary feeding diet, and a complementary feeding diet obtained or obtainable by said method.

Around the age of 6 months, the nutrient and energy requirements of a child starts to exceed what is provided by breast milk, and complementary foods are necessary to meet those needs. During the period of complementary feeding, children are at high risk of undernutrition. Complementary foods are often of inadequate nutritional quality, or they are given too early or too late, in too small amounts, or not frequently enough.

The term “complementary feeding period” may refer to the period in which complementary feeding occurs and typically covers the period from 6 months to 24 months of age. The complementary feeding period may cover from 6 months to 24 months of age or from 6 months to less than 24 months of age (e.g. 23.9 months of age).

The term “complementary feeding diet” may refer to the part of a child's diet provided by complementary foods during the complementary feeding period. The term “complementary foods” may include foods or liquids other than breast milk. Preferably, the term “complementary foods” includes foods or liquids other than breast milk, infant formula, or cow's milk.

The term “infant formula” may refer to a foodstuff intended for infants during the first year of life and satisfying by itself the nutritional requirements of this category of person, as defined in European Commission Regulation (EU) 2016/127 of 25 Sep. 2015. The term “infant formula” may also refer to a nutritional composition intended for infants and as defined in Codex Alimentarius (Codex STAN 72-1981). The term “infant formula” encompasses both “follow-up formula” and “follow-on formula” which may be given from the 6th month onwards.

The term “cow's milk” may include unfortified cow's milk and/or fortified cow's milk such as growing-up milks (GUMs). GUMs are milk-based drinks with added vitamins and minerals intended for children aged 12-36 months.

The complementary feeding diet may be determined for any child.

Suitably, the child may be aged from 6 months to 24 months, or from 6 months to less than 24 months (e.g. 23.9 months). Suitably, the child may be an infant or a toddler.

The term “infant” may refer to a child aged from 0 months to 12 months, or from 0 months to less than 12 months (e.g. 11.9 months). The child may be an infant aged from 6 months to 12 months, or from 6 months to less than 12 months (e.g. 11.9 months). The child may be an infant aged from 6 months to 9 months, or from 6 months to less than 9 months (e.g. 8.9 months) or an infant aged from 9 months to 12 months, or from 9 months to less than 12 months (e.g. 11.9 months).

The term “toddler” may refer to a child aged from 12 months to 36 months, or from 12 months to less than 36 months (e.g. 35.9 months). The child may be a toddler aged from 12 months to 24 months, or from 12 months to less than 24 months (e.g. 23.9 months).

Suitably, the child is aged from 6 months to less than 9 months, from 9 months to less than 12 months, or from 12 months to less than 24 months.

The child may be breast-fed, formula-fed, milk-fed (e.g. cow's milk fed), or any combination thereof. In some embodiments, the child is breast-fed, formula-fed, breast-fed and formula-fed, or milk-fed (e.g. cow's milk fed). Suitably, when the child is breast-fed and formula-fed, the ratio of breast milk to formula may be from about 45:55 to about 55:45, or the ratio of breast milk to formula may be about 50:50.

The child's diet may comprises the complementary feeding diet and one or more of breast milk, infant formula, and cow's milk. In some embodiments, the child's diet comprises the complementary feeding diet and breast milk; the complementary feeding diet and infant formula; the complementary feeding diet, breast milk and infant formula; or the complementary feeding diet and cow's milk. Suitably, when the child's diet comprises breast milk and infant formula, the ratio of breast milk to infant formula may be from about 45:55 to about 55:45, or the ratio of breast milk to infant formula may be about 50:50.

In some embodiments:
(i) the child is aged from 6 months to less than 9 months and the child's diet comprises the complementary feeding diet and breast milk;
(ii) the child is aged from 6 months to less than 9 months and the child's diet comprises the complementary feeding diet and infant formula;
(iii) the child is aged from 6 months to less than 9 months and the child's diet comprises the complementary feeding diet, breast milk and infant formula;
(iv) the child is aged from 9 months to less than 12 months and the child's diet comprises the complementary feeding diet and breast milk;
(v) the child is aged from 9 months to less than 12 months and the child's diet comprises the complementary feeding diet and infant formula;
(vi) the child is aged from 9 months to less than 12 months and the child's diet comprises the complementary feeding diet, breast milk and infant formula; or
(vii) the child is aged from 12 months to less than 24 months and the child's diet comprises the complementary feeding diet and cow's milk.

The infant formula and milk may be fortified or unfortified.

The proportion of the child's diet which is provided by complementary foods and non-complementary foods may be readily determined by any suitable method known in the art. For example, the ratio of non-complementary food to complementary food in the child's diet may be based on published observations (e.g. Butte N, et al. Journal of the American Dietetic Association. 2004; 104(3):442-54; and WHO. Complementary feeding of young children in developing countries: A review of current scientific knowledge. 1998).

In some embodiments, the child's diet may further comprise one or more supplements.

### Energy and Nutrient Requirements

The child's diet may meet substantially all or all the requirements of the child during the complementary feeding period. The child's diet may meet substantially all or all the nutrient and/or energy requirements of the child during the complementary feeding period.

The complementary feeding diet may be provided to meet substantially all or all the requirements of the child's diet during the complementary feeding period (in combination with the non-complementary foods which form the remainder of the child's diet). The complementary feeding diet may be provided to meet substantially all or all the nutrient and/or energy requirements of the child's diet during the complementary feeding period (in combination with the non-complementary foods which form the remainder of the child's diet).

As used herein, “substantially all” may refer all the requirements excluding one or two requirements.

Suitably, the child's diet meets all the requirements of the child during the complementary feeding period or all the requirements of the child during the complementary feeding period except one or two requirements. Suitably, the child's diet meets all the requirements of the child during the complementary feeding period or all the requirements of the child during the complementary feeding period except one requirement. Suitably, the child's diet meets all the requirements of the child during the complementary feeding period.

Suitably, the complementary feeding diet is provided to meet all the requirements of the child's diet during the complementary feeding period (in combination with the non-complementary foods which form the remainder of the child's diet) or all the requirements of the child's diet during the complementary feeding period except one or two requirements (in combination with the non-complementary foods which form the remainder of the child's diet). Suitably, the complementary feeding diet is provided to meet all the requirements of the child's diet during the complementary feeding period (in combination with the non-complementary foods which form the remainder of the child's diet) or all the requirements of the child's diet during the complementary feeding period except one requirement (in combination with the non-complementary foods which form the remainder of the child's diet). Suitably, the complementary feeding diet is provided to meet all the requirements of the child's diet during the complementary feeding period (in combination with the non-complementary foods which form the remainder of the child's diet).

### Energy Requirements

The child's diet may meet the energy requirements of the child during the complementary feeding period.

The energy requirements of the child may be determined readily by any suitable method known in the art. Suitably, the energy requirements of the child are based on the sex of the child, the age of the child, and/or the weight of the child. Suitably, the energy requirements of the child are based on the age of the child. For example:
(i) if the child is aged from 6 months to less than 9 months the energy requirements may be based on the energy requirements for a child aged from 6 months to less than 9 months;
(ii) if the child is aged from 9 months to less than 12 months the energy requirements may be based on the energy requirements for a child aged from 9 months to less than 12 months; and/or
(iii) if the child is aged from 12 months to less 24 months the energy requirements may be based on the energy requirements for a child aged from 12 months to less 24 months.

Suitably, the energy requirements of the child may be determined based on published energy requirements. For example, the energy requirements of the child may be based on WHO/FAO guidelines.

The energy requirements of the child may be based on the Estimated Energy Requirement (EER) calculated based on the sex of the child, the age of the child, and/or the weight of the child. For example, the EER may be determined based on the sex of the child and/or the age of the child, and body weight values from the Growth Chart Percentiles from the World Health Organization (WHO), for example available from WHO Multicentre Growth Reference Study Group. WHO Child Growth Standards: Length/height-for-age, weight-for-age, weight-for-length, weight-for-height and body mass index-for-age: Methods and development. Geneva: World Health Organization, 2006. The EER may be determined using the method described in Butte N F. Public Health Nutr. 2005 October; 8(7A):953-67, for example using the formula:

EER [kcal/day]=89*weight [kg]−100

Suitably, the energy requirements of the child are expressed as a minimum and/or maximum energy intake. Suitably, the energy requirements of a child's diet may be determined based on the sex of the child and/or the age of the child, and the body weight values from the 5th to 95th percentile, or the 25th to 75th percentile of the Growth Chart Percentiles from the WHO. For example:
(i) if the child is aged from 6 months to less than 9 months the energy requirements of the child may be about 584 kcal/day to about 693 kcal/day;
(ii) if the child is aged from 9 months to less than 12 months the energy requirements of the child may be about 648 kcal/day to about 771 kcal/day; or
(iii) if the child is aged from 12 months to less than 24 months the energy requirements of the child may be about 791 kcal/day to about 940 kcal/day

In some embodiments, the energy requirements of the child are isocaloric, i.e. a single energy requirement is determined for the child based on the sex of the child, the age of the child, and/or the weight of the child. The single energy requirement may be a median energy requirement, based on the sex of the child, the age of the child, and/or the weight of the child.

In some embodiments, the isocaloric energy requirements of the child are based on the age of the child. Suitably, the energy requirements of a child's diet may be determined based on the sex of the child and/or the age of the child, and body weight values from 50th percentile of the Growth Chart Percentiles from the WHO. For example, the energy requirements of a child's diet may be determined based on the age of the child and the body weight value from 50th percentile of the Growth Chart Percentiles from the WHO for boys and girls (averaged). In some embodiments:
(i) if the child is aged from 6 months to less than 9 months the energy requirements of the child may be about 636 kcal/day;
(ii) if the child is aged from 9 months to less than 12 months the energy requirements of the child may be about 706 kcal/day; or
(iii) if the child is aged from 12 months to less than 24 months the energy requirements of the child may be about 860 kcal/day.

The energy requirements can be expressed in any suitable measurement unit, for example kcal/day.

The complementary feeding diet may be provided to meet the energy requirements of the child's diet during the complementary feeding period (in combination with the non-complementary foods which form the remainder of the child's diet).

The energy provided by the non-complementary foods can be determined readily by any suitable method known in the art. For example, the proportion of energy from milk or formula may be based on published observations (e.g. WHO. Infant and young child feeding: model chapter for textbooks for medical students and allied health professionals. Geneva: WHO, 2009; Dewey K G. Pediatric Clinics of North America. 2001; 48(1):87-104; and PAHO. Guiding principles for complementary feeding of the breastfed child 2003).

Suitably, from 30% to 70% of the energy in the child's diet is provided by the complementary feeding diet. The energy provided by the non-complementary foods may be based on the age of the child, for example:
(i) if the child is aged from 6 months to less than 9 months the complementary feeding diet may provide about 30% of the energy in the child's diet, for example about 195 kcal/day;
(ii) if the child is aged from 9 months to less than 12 months the complementary feeding diet may provide about 50% of the energy in the child's diet, for example about 353 kcal/day; or
(iii) if the child is aged from 12 months to less than 24 months the complementary feeding diet may provide about 70% of the energy in the child's diet, for example about 602 kcal/day.

The energy density of non-complementary foods may be determined based on published observations. For example, the energy density of breast milk may be an average based on published observations (e.g. USDA National Nutrient Database for Standard Reference; AAP. Breastfeeding Handbook for Physicians. Schanler et al, 2006. 33 p.; and Souci/Fachmann/Kraut: Food Composition and Nutrition Tables, 8th edition). For example, the energy density of infant formula may be an average based on commercially available products. For example, the energy density of cow's milk may be based on published observations (e.g. USDA National Nutrient Database for Standard Reference). Exemplary energy densities are shown in Tables 1 and 2.

### Nutrient Requirements

The child's diet may meet substantially all or all the nutrient requirements of the child during the complementary feeding period.

The term “nutrient” may refer to any substance which is essential for growth and health of the child. The term nutrient encompasses “macronutrients”, such as carbohydrates, fats and fatty acids, and proteins and “micronutrients”, such as vitamins and minerals.

Exemplary macronutrients include carbohydrates (including fibre and sugars), protein, and lipids (including long chain polyunsaturated fatty acids).

Exemplary micronutrients include vitamins (including vitamin A, vitamin D, vitamin C, folate, vitamin B6, vitamin B12, and vitamin E) and minerals (including sodium, potassium, calcium, iron, zinc, magnesium, and phosphorus).

The nutrient requirements of the child may be determined readily by any suitable method known in the art. Suitably, the nutrient requirements of the child are based on the sex of the child, the age of the child, and/or the weight of the child. Suitably, the nutrient requirements of the child are based on the age of the child. For example:
(i) if the child is aged from 6 months to less than 9 months the nutrient requirements may be based on the nutrient requirements for a child aged from 6 months to less than 9 months;
(ii) if the child is aged from 9 months to less than 12 months the nutrient requirements may be based on the nutrient requirements for a child aged from 9 months to less than 12 months; and/or
(iii) if the child is aged from 12 months to less 24 months the nutrient requirements may be based on the nutrient requirements for a child aged from 12 months to less 24 months.

Suitably, the nutrient requirements of the child may be determined based on published nutrient requirements. For example, the nutrient requirements of the child may be based on WHO/FAO guidelines, ESPGHAN guidelines, and/or Board IOM FaN Dietary Reference Intakes.

The nutrient requirements may take into account bioavailability for one or more nutrient. For example, the nutrient requirements may take into account the bioavailability of vitamins and minerals such as iron, zinc, and calcium. In some embodiments, the nutrient requirements for iron takes into account bioavailability of iron. Exemplary values for the bioavailability of iron are shown in Table 3.

The nutrient requirements can be expressed in any suitable measurement unit. For example, macronutrients can be expressed in grams per day (g/d) or as a percentage of total energy (% E); micronutrients can be expressed in milligrams per day (mg/d) or micrograms per day (mcg/d).

There may be more than one nutrient requirement for each nutrient. Suitably, the nutrient requirements may include a minimum and/or maximum nutrient intake per day for each nutrient. Suitably, the nutrient requirements may be expressed in two or more measurement units e.g. both as a total amount per day and/or as a percentage of total energy per day. Suitably, the nutrient requirements may include a requirement taking into account bioavailability and a requirement not taking into account bioavailability, e.g. for iron.

Exemplary nutrient requirements (minimum and/or maximum intake) are provided in FIGS. 4, 5D, 6D, and 7D.

The complementary feeding diet may be provided to meet substantially all or all the nutrient requirements of the child's diet during the complementary feeding period (in combination with the non-complementary foods which form the remainder of the child's diet).

The nutrients provided by the non-complementary foods can be determined readily by any suitable method known in the art. For example, the nutrient composition of breast milk may be an average based on published observations (e.g. USDA National Nutrient Database for Standard Reference; AAP. Breastfeeding Handbook for Physicians. Schanler et al, 2006. 33 p.; and Souci/Fachmann/Kraut: Food Composition and Nutrition Tables, 8th edition). For example, the nutrient composition of infant formula may be an average based on commercially available products. For example, the nutrient composition of cow's milk may be based on published observations (e.g. USDA National Nutrient Database for Standard Reference). Exemplary nutrient compositions are shown in Tables 1 and 2. The volume of milk may be based on the proportion of energy from milk and the energy density of the milk, as described above.

### Food Types

The complementary feeding diet may provide a plurality of food groups.

The term “food group” may refer to a collection of foods that share similar nutritional properties or biological classifications. Nutrition guides typically divide foods into food groups and Recommended Dietary Allowance recommend daily servings of each group for a healthy diet. Exemplary food groups include fruits; vegetables; pulses, nuts or seeds; meats; starches or grains; dairy; and oils and fats.

The complementary feeding diet may also provide a plurality of food types.

The term “food type” may refer to a collection of foods from the same food group that share more similar nutritional properties or biological classifications. Each food group may be further grouped into a plurality of food types. Exemplary food types for the food group fruit can include apples, banana, citrus, berries, other fruits (e.g. pear, peach, pineapple), and dried fruits.

Suitable food groups and food types can be readily determined by any suitable method known in the art. For example, suitable food groups and food types can be based on published observations (e.g. Dwyer J T. The Journal of Nutrition. 2018; 148(suppl 3):1575S-80S).

In some embodiments, the complementary feeding diet provides a plurality of food types comprising at least: one or more types of fruit; one or more types of vegetable; one or more types of pulse, nut or seed; one or more types of meat; one or more types of starch or grain; one or more type of dairy; and one or more type of oil and fat.

The food group “fruit” may comprise any type of fruit which is suitable as a complementary food, such as apples, banana, citrus, berries, pear, peach, pineapple, and dried fruits. In some embodiments, the one or more types of fruit comprise one or more of: apples, banana, citrus, berries, other fruits (e.g. pear, peach, pineapple), and dried fruits.

The food group “vegetables” may comprise any type of vegetable which is suitable as a complementary food, such as carrots, broccoli, spinach, sweet potato, peas, cauliflower, cabbage, green beans, zucchini, and avocado. In some embodiments, the one or more types of vegetables comprises one or more of: orange/red vegetables (e.g. carrots), dark green vegetables (e.g. broccoli, spinach), starchy vegetables (e.g. sweet potato, peas), other vegetables (e.g. cauliflower, cabbage, green beans, zucchini), and avocado.

The food group “pulses, nuts or seeds” may comprise any type of pulse, nut or seed which is suitable as a complementary food, such as dried beans, peas, legumes, and flax seed. In some embodiments, the one or more types of pulse, nut or seed comprises one or more of: dried beans/peas/legumes, and nuts/seeds.

The food group “meats” may comprise any type of meat which is suitable as a complementary food, such as beef, chicken, turkey, eggs, lamb, goat, game, pork, fish, shellfish, organ meats, bacon, sausage, and cold cuts. In some embodiments, the one or more types of meat comprises one or more of: beef, chicken/turkey, eggs, lamb/goat/game, pork, fish/shellfish, organ meats, and bacon/sausage/cold cuts.

The food group “starches or grains” may comprise any type of starch or grain which is suitable as a complementary food, such as infant cereal, oat meal, polenta, white potato, rice, pasta, white bread, wholegrain bread, rice cakes, crackers, and unsweetened RTE cereal. In some embodiments, the one or more types of starches or grains comprises one or more of: infant cereal, hot cereal, white potato, rice/pasta, white bread/rolls/tortilla/bagel, wholegrain bread/rolls/tortilla/bagel, rice cakes/pretzels/crackers, and unsweetened ready-to-eat cereal.

The food group “dairy” may comprise any type of dairy product which is suitable as a complementary food, such as yogurt or cheese. Preferably, the food type “dairy” excludes milk products such as breast milk, infant formula or cow's milk. In some embodiments, the one or more types of dairy comprises one or more of: yogurt and cheese.

The food group “oils and fats” may comprise any type of oil or fat which is suitable as a complementary food, such as butter, margarine, cream, oil, dressing, and mayonnaise. In some embodiments, the one or more types of oil and fat comprise one or more of: butter, margarine, cream, oil, dressing, and mayonnaise.

The energy nutrients provided by each food group and/or food type can be determined readily by any suitable method known in the art. For example, the nutrient composition for each food type can be an average based on published observations (e.g. Nutrition Data System for Research (NDSR, version 2015: University of Minnesota, Minneapolis, MN)).

Complementary foods and non-complementary foods with any level of fortification may be used in the method of the invention. The complementary feeding diet may be fortified or unfortified.

Food fortification or enrichment is the process of adding micronutrients (essential trace elements and vitamins) to food. For example, most wheat-based products in the US are fortified with iron, folic acid, and other B vitamins and cow's milk may be fortified with vitamins A and D.

In some embodiments, the complementary feeding diet is fortified. In some embodiments, the complementary feeding diet is unfortified.

The energy nutrients provided by each fortified or unfortified food group and/or fortified or unfortified food type can be determined readily by any suitable method known in the art. For example, the macronutrient composition for fortified or unfortified food types may be the same and the micronutrient composition for fortified or unfortified food types may be based on published observations (e.g. ANSES. CIQUAL food composition table 2017).

In some embodiments, the complementary feeding diet is diverse.

As used herein, a “diverse complementary feeding diet” provides at least 4 food groups per day. Suitably, a diverse complementary feeding diet comprises at least 5 food groups per day, or at least 6 food groups per day, or at least 7 food groups per day. Preferably, a diverse complementary feeding diet provides at least 7 food groups per day. Studies have found that a higher dietary diversity is associated with better nutritional status.

In some embodiments, the complementary feeding diet is age-appropriate.

As used herein, an “age-appropriate complementary feeding diet” is one which only provides food types which are age or developmentally appropriate. Food types which are age or developmentally appropriate will be well known to the skilled person and may be based on published guidance (e.g. B Altkorn G R, et al. Encyclopedia of Food Safety, Volume 3. Waltham, MA: Academic Press; pp 110-6). For example, choking hazards for infants may include raw carrots, raw apple, etc.

In some embodiments, the complementary feeding diet is palatable.

As used herein, a “palatable complementary feeding diet” may refer to a complementary feeding diet which will be readily accepted by the child. The skilled person will be able to readily determine whether a diet will be readily accepted by the child and may be based on published guidance.

In some embodiments, the complementary feeding diet is realistic.

As used herein, a “realistic complementary feeding diet” may refer to a complementary feeding diet which resembles an average complementary feeding diet for the child. For example, the diet does not deviate substantially from an average complementary feeding diet. A realistic complementary feeding diet can be transferable to a realistic menu for the child.

In some embodiments, the complementary feeding diet is generalisable.

As used herein, a “generalisable complementary feeding diet” may refer to a complementary feeding diet which can generalised for different children. For example, a generalisable complementary feeding diet may be generalisable for boys and girls, across a number or regions or countries, and/or regardless of special diets. A generalisable complementary feeding diet can be transferable to a generalisable menu for the child.

The complementary feeding diet may be diverse, age-appropriate, palatable, realistic, and generalisable, or any combination thereof. Any suitable constraints can be used to ensure that a complementary feeding diet is obtained which is diverse, age-appropriate, palatable, realistic and/or generalisable.

In some embodiments, the complementary feeding diet provides a plurality of food groups comprising at least: fruits; vegetables; pulses, nuts or seeds; meats; starches or grains; dairy; and oils and fats.

In some embodiments, the complementary feeding diet provides at least: one or more types of fruit; one or more types of vegetable; one or more types of pulse, nut or seed; one or more types of meat; one or more types of starch or grain; one or more type of dairy; and one or more type of oil and fat.

In some embodiments, the amount of each food group and/or food type provided by the complementary feeding diet does not exceed a maximum intake. The maximum intake of each food group and/or food type may be readily determined by any suitable method known in the art. Suitably, the maximum intake is based on the sex of the child, the age of the child, and/or the weight of the child. Suitably, the maximum intake is based on the age of the child. Suitably, the maximum intake may be determined based on published intakes. For example, the maximum intake for each food group and/or food type may be based on, separately for each age group, the 95th percentile of the distribution among FITS intakes (e.g. Dwyer J T. The Journal of Nutrition. 2018; 148(suppl 3):1575S-80S).

In some embodiments, the complementary feeding diet meets at least one or more of the following criteria:
(a) at least 10 g/day of two types of fruit;
(b) at least 10 g/day of three types of vegetable;
(c) no more than 50 g/day egg and no more than 150 g/week egg;
(d) no more than 21 g/day of any one type of meat; and
(e) a limit of 10% total energy from occasional foods.

In some embodiments, the complementary feeding diet meets all of the following criteria:
(a) at least 10 g/day of two types of fruit;
(b) at least 10 g/day of three types of vegetable;
(c) no more than 50 g/day egg and no more than 150 g/week egg;
(d) no more than 21 g/day of any one type of meat; and
(e) optionally, a limit of 10% total energy from occasional foods.

In some embodiments, the plurality of food types excludes one or more food types. Suitably, the one or more excluded food types comprise one or more of:
(a) foods with low or no nutrient contribution;
(b) developmentally or age inappropriate foods;
(c) occasional foods, for example 100% fruit juice;
(d) supplements;
(e) foods for special diets;
(f) regional-specific foods; and
(g) mixed foods.

In some embodiments, the plurality of food types excludes all of:
(a) foods with low or no nutrient contribution;
(b) developmentally or age inappropriate foods;
(c) occasional foods, for example 100% fruit juice;
(d) supplements;
(e) foods for special diets;
(f) regional-specific foods; and
(g) mixed foods.

“Foods with low or no nutrient contribution” can be readily determined by the skilled person and may include, for example, condiments such as ketchup and mustard.

“Developmentally or age inappropriate foods” may be readily determined by the skilled person (e.g. based on published guidelines) and may include, for example, choking hazards (such as raw carrot, raw apple, sausages, hot dogs, popcorn, hard candy), RTE cereal, and tea/coffee.

“Occasional foods” are foods with high energy and low nutrient diet and may include, for example, sweet and salty snack foods, French fries, and 100% fruit juice.

“Foods for special diets” are foods which are designed for special diets, such as vegetarian or vegan diets and may include, for example plant-based milks, egg substitutes, and vegetarian meats. These may be excluded to provide a generalisable complementary feeding diet for children consuming an omnivorous diet.

“Regional-specific foods” are foods which are specific for a certain country or region. For example, US-specific foods may include pancakes, French toast, biscuits and peanut butter. These may be excluded to ensure the complementary feeding diet is generalisable across a number or regions or countries.

“Mixed foods” are foods which comprise one or more food type such as mixed dishes, mixed vegetable, mixed fruits, cereal bars and the like.

Any other food type may be excluded as necessary. For example, commercial baby foods such as fruit purees and baby food dinners may be excluded to ensure the complementary feeding diet is generalisable.

The excluded food types may be based on age. For example, white bread may be inappropriate for infants aged from 6 months to less than 9 months, but appropriate for infants aged 9 months or older. For example, sausages may be inappropriate for infants aged less than 12 months, but appropriate for infants aged 12 months or older. In some embodiments, occasional foods are excluded for infants aged less than 12 months, but not excluded for infants aged 12 months or older provided the complementary feeding diet meets a limit of 10% total energy from occasional foods.

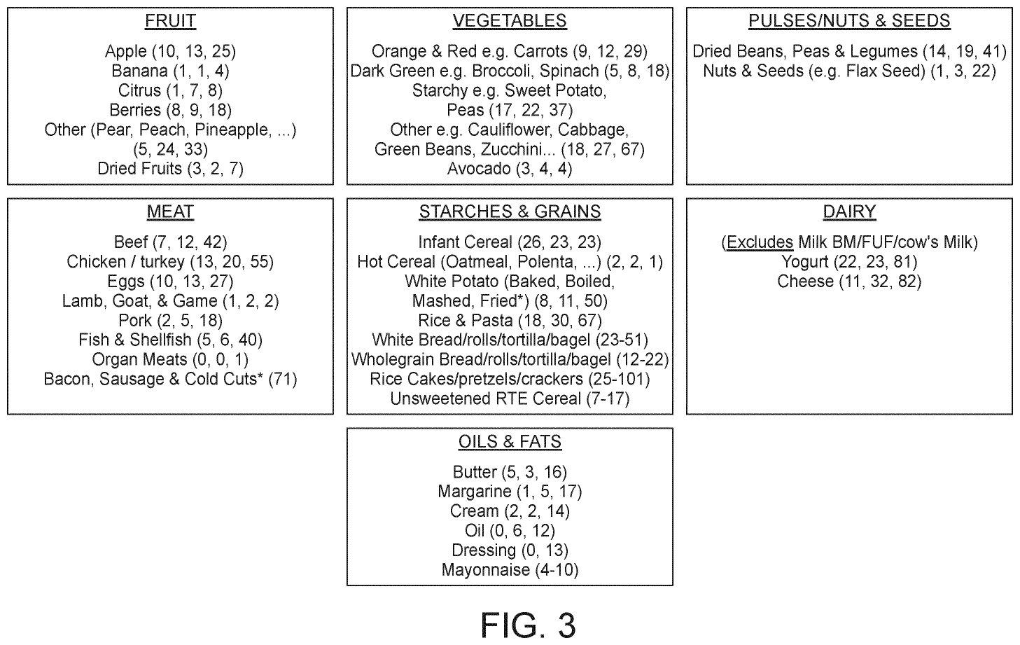

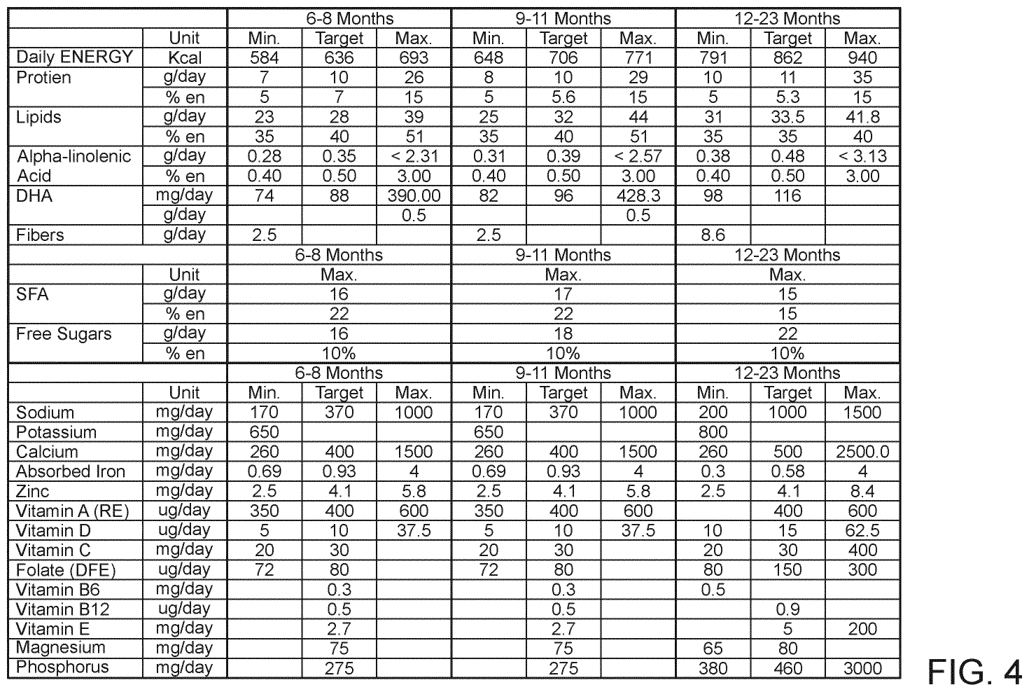

Exemplary food groups and food types (by age) are shown in FIG. 3.

### Method for Determining a Complementary Feeding Diet

The complementary feeding diet may be determined by any method described herein.

The complementary feeding diet may be determined for any suitable duration. For example, the complementary feeding diet may be determined for a period of one week, since nutrient requirements are typically intended to be achieved within such a period. Suitably, the complementary feeding diet is determined for a period of at least one week, at least two weeks, at least three weeks, or at least four weeks. In some embodiments, the complementary feeding diet is a weekly complementary feeding diet. The complementary diet can be transferable to a corresponding menu for the child. For example, a weekly complementary feeding diet can be transferable to a weekly menu for the child.

The complementary feeding diet may be based on nutrient (and optionally energy) targets and/or average intakes. Determining the complementary feeding diet based on nutrient (and optionally energy) targets can provide an optimised complementary feeding diet to meet said targets. Determining the complementary feeding diet based on average intakes can provide a palatable and realistic complementary feeding diet.

In some embodiments, the complementary feeding diet is determined by: (a) providing (i) targets for a plurality of nutrients (and optionally energy) and/or (ii) average intakes for the plurality of food types; and (b) determining the complementary feeding diet by minimising (i) the (negative) deviation between the nutrient targets (and optionally energy target) and the amount of each nutrient (and optionally energy) in the child's diet and/or (ii) the deviation between the average intakes and the amount of each food type in the child's diet.

### Nutrient Targets

As described above, the complementary feeding diet may be provided to meet substantially all or all the nutrient requirements of the child's diet during the complementary feeding period (in combination with the non-complementary foods which form the remainder of the child's diet). In addition, the complementary feeding diet may be optimised by minimising the (negative) deviation between a plurality of nutrient targets (e.g. which lie within the nutrient requirements of the child's diet) and the amount of each nutrient in the child's diet.

In some embodiments, the complementary feeding diet is determined by: (a) providing targets for a plurality of nutrients; and (b) determining the complementary feeding diet by minimising the (negative) deviation between the nutrient targets and the amount of each nutrient in the child's diet.

Targets can be provided for any suitable nutrient. Suitably, nutrient targets are provided for at least five, at least six, at least seven, at least eight, at least nine, at least ten, at least eleven, or at least twelve nutrients.

Nutrient targets can be provided for nutrients for which the nutrient requirements are typically exceeded or inadequate in a child's diet. Such nutrients can be readily determined by the skilled person, for example based on average nutrient intakes for such a child. Nutrient targets for other nutrients (i.e. nutrients for which the nutrient requirements are typically met) need not be included because these are likely to be met provided the other nutrient targets are met.

Such nutrients can be readily identified by any method known in the art, for example by sensitivity analysis.

The nutrient targets may comprise targets for at least protein, lipids, iron, calcium, and zinc.

The nutrient targets may further comprise targets for long chain polyunsaturated fatty acids, for example alpha-linolenic acid (ALA) and/or docosahexaenoic acid (DHA). Preferably, the nutrient targets further comprise targets for ALA and DHA.

The nutrient targets may further comprise targets for one or more minerals, for example for magnesium, phosphorus, potassium, and sodium. Preferably, the nutrient targets further comprise targets for magnesium and phosphorus. More preferably, the nutrient targets further comprise targets for magnesium, phosphorus, potassium, and sodium.

The nutrient targets may further comprise targets for one or more vitamins, for example vitamin A, vitamin C, folate, vitamin D, vitamin B6, vitamin B12, and vitamin E. Preferably, the nutrient targets further comprise targets for vitamin A, vitamin C, and folate. More preferably, the nutrient targets further comprise targets for vitamin A, vitamin C, folate, vitamin D, vitamin B6, vitamin B12, and vitamin E.

In some embodiments, the nutrient targets comprise targets for at least protein, lipids, ALA, DHA, iron, calcium, zinc, vitamin A, vitamin C, and folate, and optionally potassium, sodium, magnesium, phosphorus, vitamin D, vitamin B6, vitamin B12, and vitamin E.

In some embodiments:
(i) the child is aged from 6 months to less than 9 months and the nutrient targets comprise targets for at least protein, lipids, ALA, DHA, iron, calcium, zinc, sodium, potassium, vitamin A, vitamin C, and folate;
(ii) the child is aged from 9 months to less than 12 months and the nutrient targets comprise targets for at least protein, lipids, ALA, DHA, iron, calcium, zinc, sodium, potassium, phosphorous, vitamin A, vitamin C, folate, vitamin D, vitamin B6, vitamin B12, and vitamin E; and/or
(iii) the child is aged from 12 months to less 24 months and the nutrient targets comprise targets for at least protein, lipids, ALA, DHA, iron, calcium, zinc, sodium, magnesium, phosphorus, vitamin A, vitamin C, folate, and vitamin B6.

The nutrient targets of the child may be determined by any suitable method known in the art. Suitably, the nutrient targets of the child are based on the sex of the child, the age of the child, and/or the weight of the child. Suitably, the nutrient targets of the child are based on the age of the child. For example:
(i) if the child is aged from 6 months to less than 9 months the nutrient targets may be based on the nutrient targets for a child aged from 6 months to less than 9 months;
(ii) if the child is aged from 9 months to less than 12 months the nutrient targets may be based on the nutrient targets for a child aged from 9 months to less than 12 months; and/or
(iii) if the child is aged from 12 months to less 24 months the nutrient targets may be based on the nutrient targets for a child aged from 12 months to less 24 months.

Suitably, the nutrient targets of the child may be determined based on published nutrient targets. For example, the nutrient targets of the child may be based on WHO/FAO guidelines, ESPGHAN guidelines, and/or Board IOM FaN Dietary Reference Intakes.

The nutrient targets may take into account bioavailability for one or more nutrient. For example, the nutrient targets may take into account the bioavailability of vitamins and minerals such as iron, zinc, and calcium. Preferably, the nutrient targets for iron takes into account bioavailability of iron. Exemplary values for the bioavailability of iron shown in Table 3. Nutrient targets taking into account bioavailability need not be included if these are likely to be met provided the other nutrient targets are met. This can be readily determined by any method known in the art, for example by sensitivity analysis.

The nutrient targets can be expressed in any suitable measurement unit. For example, macronutrients can be expressed in grams per day (g/d) or as a percentage of total energy (% E); micronutrients can be expressed in milligrams per day (mg/d) or micrograms per day (mcg/d).

There may be one than one nutrient target for each nutrient. Suitably, the nutrient targets may be expressed in two or more measurement units e.g. both as a total amount per day and/or as a percentage of total energy per day. Suitably, the nutrient target may include a target taking into account bioavailability and a target not taking into account bioavailability, e.g. for iron.

Exemplary nutrient targets are provided in FIGS. 4, 5D, 6D, and 7D.

### Energy Target

As described above, the complementary feeding diet may be provided to meet the energy requirements of the child's diet during the complementary feeding period (in combination with the non-complementary foods which form the remainder of the child's diet). In addition, the complementary feeding diet may be optimised by minimising the (negative) deviation between an energy target (e.g. a target which lies within the energy requirements of the child's diet) and the amount of energy in the child's diet.

In some embodiments, the targets may include an energy target, in addition to the plurality of nutrient targets.

For example, the complementary feeding diet may be determined by: (a) providing targets for a plurality of nutrients and energy; and (b) determining the complementary feeding diet by minimising the (negative) deviation between the nutrient targets and energy target and the amount of each nutrient and energy in the child's diet.

The energy target of the child may be determined by any suitable method known in the art, suitably using any method described above for energy requirements. Suitably, the energy target of the child is based on the sex of the child, the age of the child, and/or the weight of the child. Suitably, the energy target of the child is based on the age of the child. For example,
(i) if the child is aged from 6 months to less than 9 months the energy target of the child may be about 636 kcal/day;
(ii) if the child is aged from 9 months to less than 12 months the energy target of the child may be about 706 kcal/day; or
(iii) if the child is aged from 12 months to less than 24 months the energy target of the child may be about 860 kcal/day.

The energy target can be expressed in any suitable measurement unit, for example kcal/day.

When the energy requirements of the child are isocaloric, the energy target may not be required.

In some embodiments, the deviation between the nutrient targets (and optionally energy target) and the amount of each nutrient (and optionally energy) in the child's diet is a negative deviation, i.e. the deviation below the target. The negative deviation may be used because exceeding the nutrient target is appropriate (as long as the amount of the nutrient does not exceed the nutrient requirements).

In some embodiments, the deviation between the nutrient targets (and optionally energy target) and the amount of each nutrient (and optionally energy) in the child's diet is an average deviation, i.e. mean deviation. The average deviation may be a weighted average deviation such that some nutrients (and optionally energy) are prioritised. Any suitable weighting can be used and can be readily determined by the skilled person. For example, energy may be given a lower weighting than nutrients and/or sodium may be given a lower weighting than other nutrients since the adequate intake of sodium may not be a target that has to be met.

### Average Intakes

The complementary feeding diet may be optimised by minimising the deviation between the average intakes and the amount of each food group or food type in the child's diet.

In some embodiments, the complementary feeding diet is determined by: (a) providing average intakes for a plurality of food groups or food types; and (b) determining the complementary feeding diet by minimising the deviation between the average intakes and the amount of each food group or food type in the child's diet.

In some embodiments, the complementary feeding diet is determined by: (a) providing average intakes for a plurality of food types; and (b) determining the complementary feeding diet by minimising the deviation between the average intakes and the amount of each food type in the child's diet.

Average intakes can be provided for any food group or food type. The plurality of food groups or food types for which average intakes are provided can be any suitable food group of food type. Suitably, average intakes are provided for at least seven food groups. Suitably, average intakes are provided for at least ten, at least fifteen, at least twenty, or at least twenty-five food types.

The average intakes may be provided for at least: one or more types of fruit; one or more types of vegetable; one or more types of pulse, nut or seed; one or more types of meat; one or more types of starch or grain; one or more type of dairy; and one or more type of oil and fat.

In some embodiments, average intakes are provided for at least:
one or more of: apples, banana, citrus, berries, other fruits (e.g. pear, peach, pineapple), and dried fruits;
one or more of: orange/red vegetables (e.g. carrots), dark green vegetables (e.g. broccoli, spinach), starchy vegetables (e.g. sweet potato, peas), other vegetables (e.g. cauliflower, cabbage, green beans, zucchini), and avocado;
one or more of: dried beans/peas/legumes, and nuts/seeds;
one or more of: beef, chicken/turkey, eggs, lamb/goat/game, pork, fish/shellfish, organ meats, bacon/sausage/cold cuts;
one or more of: infant cereal, hot cereal, white potato, rice/pasta, white bread/rolls/tortilla/bagel, wholegrain bread/rolls/tortilla/bagel, rice cakes/pretzels/crackers, unsweetened ready-to-eat (RTE) cereal;
one or more of: yogurt, cheese; and
one or more of: butter, margarine, cream, oil, dressing, mayonnaise.

In some embodiments, average intakes are provided for at least:
apples, banana, citrus, berries, other fruits (e.g. pear, peach, pineapple), and dried fruits;
orange/red vegetables (e.g. carrots), dark green vegetables (e.g. broccoli, spinach), starchy vegetables (e.g. sweet potato, peas), other vegetables (e.g. cauliflower, cabbage, green beans, zucchini), and avocado;
dried beans/peas/legumes, and nuts/seeds;
beef, chicken/turkey, eggs, lamb/goat/game, pork, fish/shellfish, and organ meats, optionally bacon/sausage/cold cuts;
infant cereal, hot cereal, white potato, and rice/pasta, optionally white bread/rolls/tortilla/bagel, optionally wholegrain bread/rolls/tortilla/bagel, optionally rice cakes/pretzels/crackers, optionally unsweetened ready-to-eat (RTE) cereal;
yogurt and cheese; and
butter, margarine, cream, and oil, optionally dressing, optionally mayonnaise.

In some embodiments:
(i) the child is aged from 6 months to less than 9 months and average intakes are provided for at least: apples, banana, citrus, berries, other fruits (e.g. pear, peach, pineapple), and dried fruits; orange/red vegetables (e.g. carrots), dark green vegetables (e.g. broccoli, spinach), starchy vegetables (e.g. sweet potato, peas), other vegetables (e.g. cauliflower, cabbage, green beans, zucchini), and avocado; dried beans/peas/legumes, and nuts/seeds; beef, chicken/turkey, eggs, lamb/goat/game, pork, fish/shellfish, and organ meats; infant cereal, hot cereal, white potato, and rice/pasta; yogurt and cheese; and butter, margarine, cream, and oil;
(ii) the child is aged from 9 months to less than 12 months and average intakes are provided for at least apples, banana, citrus, berries, other fruits (e.g. pear, peach, pineapple), and dried fruits; orange/red vegetables (e.g. carrots), dark green vegetables (e.g. broccoli, spinach), starchy vegetables (e.g. sweet potato, peas), other vegetables (e.g. cauliflower, cabbage, green beans, zucchini), and avocado; dried beans/peas/legumes, and nuts/seeds; beef, chicken/turkey, eggs, lamb/goat/game, pork, fish/shellfish, and organ meats; infant cereal, hot cereal, white potato, and rice/pasta, white bread/rolls/tortilla/bagel, wholegrain bread/rolls/tortilla/bagel, rice cakes/pretzels/crackers, unsweetened ready-to-eat (RTE) cereal; yogurt and cheese; and butter, margarine, cream, oil, dressing, and mayonnaise; and/or
(iii) the child is aged from 12 months to less 24 months and average intakes are provided for at least apples, banana, citrus, berries, other fruits (e.g. pear, peach, pineapple), and dried fruits; orange/red vegetables (e.g. carrots), dark green vegetables (e.g. broccoli, spinach), starchy vegetables (e.g. sweet potato, peas), other vegetables (e.g. cauliflower, cabbage, green beans, zucchini), and avocado; dried beans/peas/legumes, and nuts/seeds; beef, chicken/turkey, eggs, lamb/goat/game, pork, fish/shellfish, organ meats, and bacon/sausage/cold cuts; infant cereal, hot cereal, white potato, and rice/pasta, white bread/rolls/tortilla/bagel, wholegrain bread/rolls/tortilla/bagel, rice cakes/pretzels/crackers, unsweetened ready-to-eat (RTE) cereal; yogurt and cheese; and butter, margarine, cream, oil, dressing, and mayonnaise.

The average intakes may be determined by any suitable method known in the art. Suitably, the average intake is a median intake or a mean intake. In some embodiments the average intake is a mean intake. Suitably, the average intakes are based on the sex of the child, the age of the child, and/or the weight of the child. Suitably, the average intakes are based on the age of the child. For example:
(i) if the child is aged from 6 months to less than 9 months the average intakes may be based on the average intakes for a child aged from 6 months to less than 9 months;
(ii) if the child is aged from 9 months to less than 12 months the average intakes may be based on the average intakes for a child aged from 9 months to less than 12 months; and/or
(iii) if the child is aged from 12 months to less 24 months the average intakes may be based on the average intakes for a child aged from 12 months to less 24 months.

Suitably, the average intakes of the child may be determined based on published intakes. For example, the average intakes may be based on Global Feeding Infants and Toddlers Studies (e.g. Roess A A, et al. The Journal of Nutrition. 2018; 148(suppl_3):1525S-35S).

The average intakes can be expressed in any suitable measurement unit. For example, average intake can be expressed in grams per day (g/d).

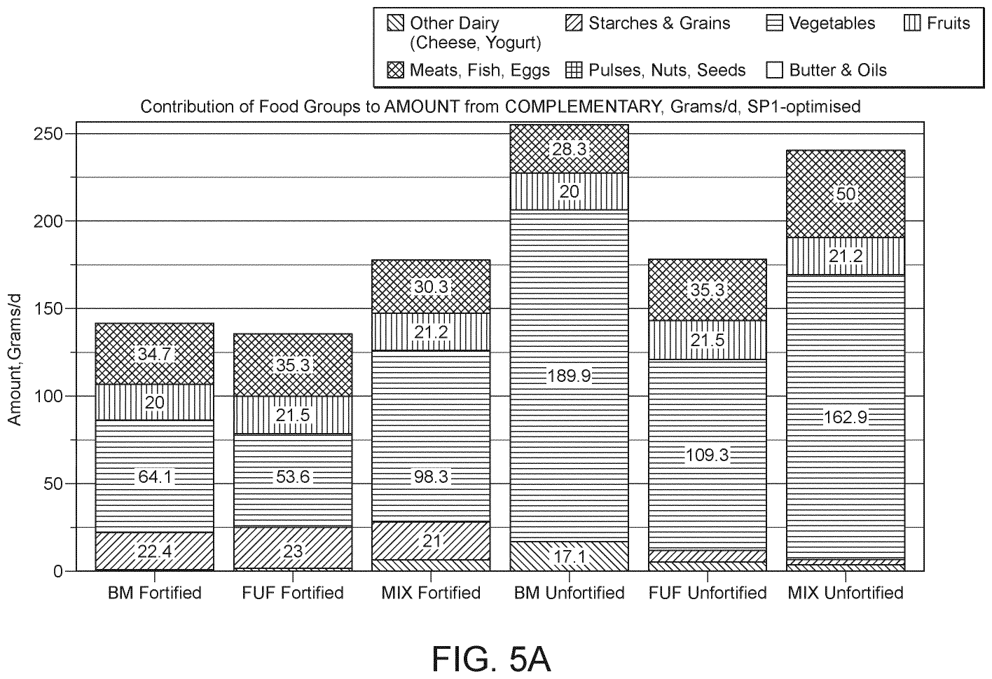

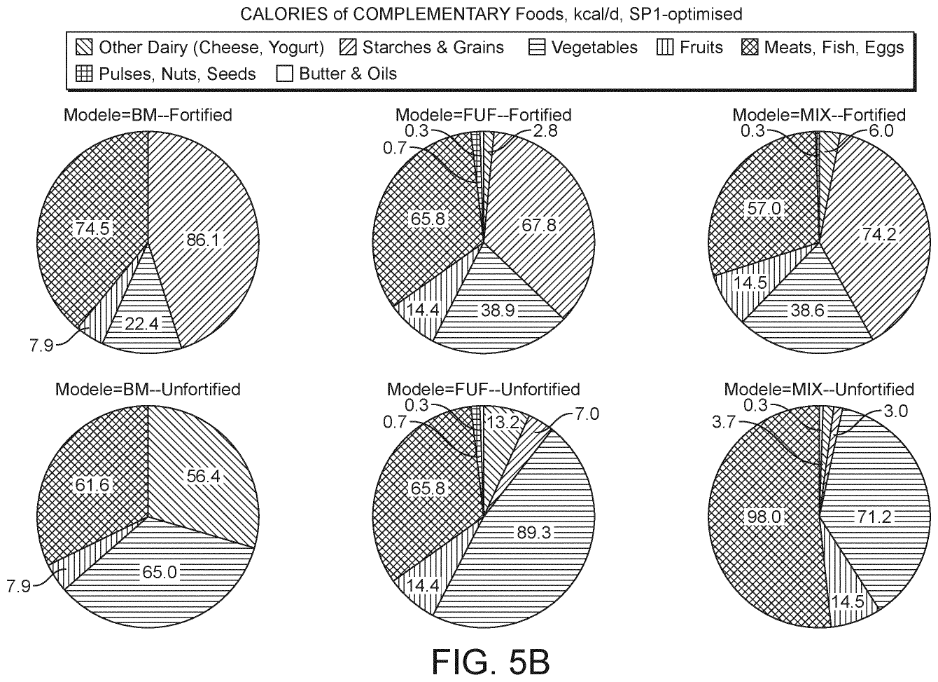

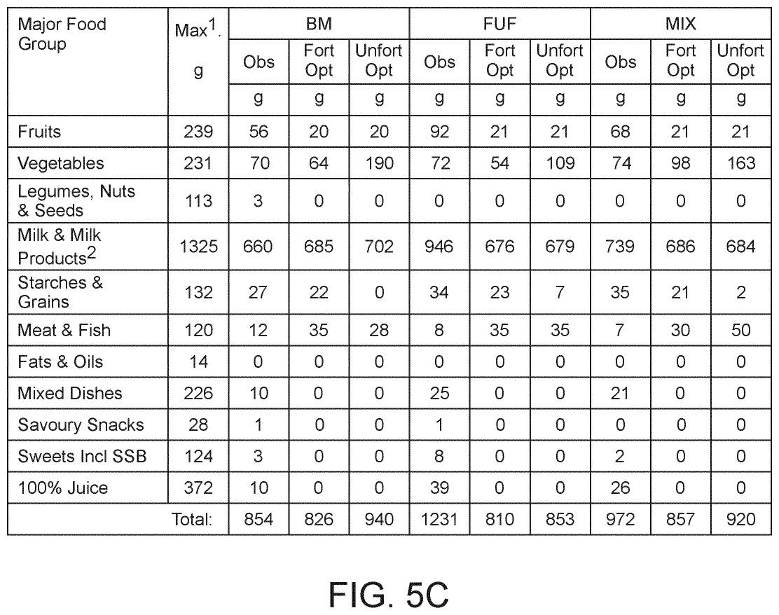

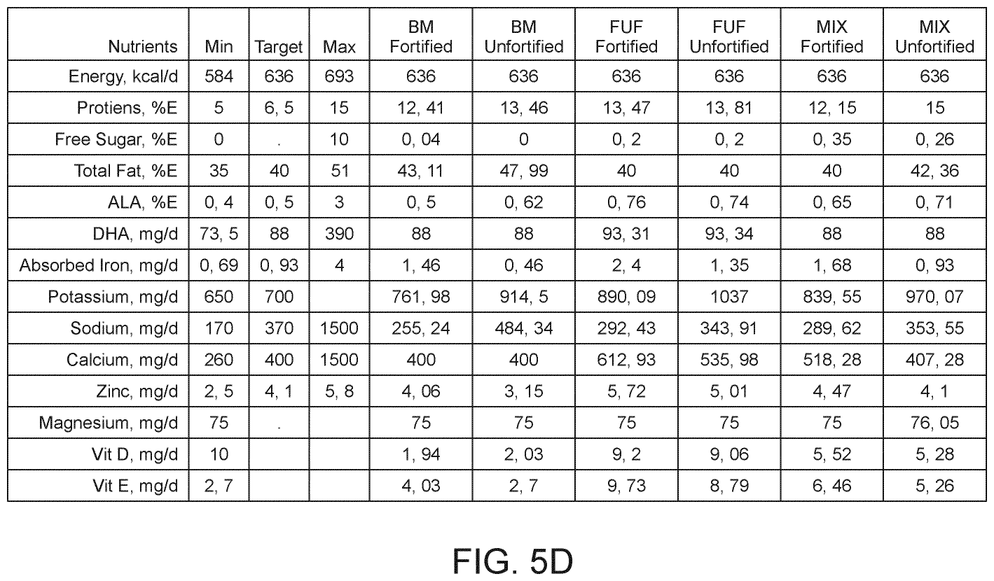

Exemplary observed average intakes (by age group) are provided in FIGS. 5C, 6C, and 7C.

In some embodiments, the deviation between the average intakes and the amount of each food type in the child's diet is an average deviation, i.e. mean deviation. The average deviation may be a weighted average deviation such that some food types are prioritised. Any suitable weighting can be used and can be readily determined by the skilled person.

### Weighted Average

In some embodiments, the complementary feeding diet is determined by: (a) providing (i) targets for a plurality of nutrients (and optionally energy) and (ii) average intakes for the plurality of food types; and (b) determining the complementary feeding diet by minimising the weighted sum of (i) the average (negative) deviation between the nutrient targets (and optionally energy target) and the amount of each nutrient (and optionally energy) in the child's diet and (ii) the average deviation between the average intakes and the amount of each food type in the child's diet.

Using both the nutrient (and optionally energy) targets and average intakes can provide an optimised complementary feeding that is palatable and realistic.

In some embodiments, the average deviation between the nutrient targets (and optionally energy target) and the amount of each nutrient (and optionally energy) in the child's diet is a negative deviation, i.e. the deviation below the target. The negative deviation may be used because exceeding the nutrient target is appropriate (as long as the amount of the nutrient does not exceed the nutrient requirements).

The weighted sum of (i) the average (negative) deviation between the nutrient targets (and optionally energy target) and the amount of each nutrient (and optionally energy) in the child's diet and (ii) the average deviation between the average intakes and the amount of each food type in the child's diet can be readily determined by any suitable method. For example, both average (i.e. mean) deviations may be expressed as a percentage, then the weighting applied, and then the weighted average deviations (expressed as a percentage) may be summed.

The weighting can be any suitable weighting and can be applied to either one or both average deviations as a whole, or to each target individually, or to each average intake individually, or any combination thereof.

For example, if the weighting is applied to both average deviations as a whole, the weighted sum may be expressed as the following formula:

$${W}_{1}^{⋆} {AVE\_DEV}_{1} + {W}_{2}^{⋆} {AVE\_DEV}_{2}$$

where: AVE_DEV₁=the average (negative) deviation between the nutrient targets (and optionally energy target) and the amount of each nutrient (and optionally energy) in the child's diet, expressed as a percentage; AVE_DEV₂=the average deviation between the average intakes and the amount of each food type in the child's diet, expressed as a percentage; W₁ is the weighting given to AVE_DEV₁; and W₂ is the weighting given to AVE_DEV₂.

W₁ and W₂ may be take any suitable positive values, depending on which deviation is prioritised. For example, if the priority is to provide an optimised complementary feeding diet then the weighting for W₁ should be greater than the weighting for W₂, whereas if the priority is to provide a palatable and realistic complementary feeding diet then the weighting for W₂ should be greater than the weighting for W₁.

In some embodiments, W₁ is greater than W₂. Suitably, W₁ is at least 10 times greater than W₂, at least 100 times greater than W₂, at least 1000 times greater than W₂. In some embodiments, W₁ is 1000 times greater than W₂. Suitably, W₁≥10 and W₂=1; W₁≥100 and W₂=1; or W₁≥1000 and W₂=1. In some embodiments, W₁=1000 and W₂=1.

For example, if the weighting is applied to each target individually, the weighted sum may be expressed as the following formula:

$$\frac{{\sum }_{f = 1}^{f = RF} \vert \frac{{Q}_{f}^{opt} - {Q}_{f}^{obs}}{{Q}_{f}^{obs}} \vert + {\sum }_{f = 1}^{f = NRF} \vert \frac{{Q}_{f}^{opt}}{{Q}_{f}^{average}} \vert}{R F + N R F} + \frac{{\sum }_{n = 1}^{n = N} {w}_{n} \vert \frac{{nutrient}_{n}^{opt} - {Nutrient}_{n}^{Target}}{{Nutrient}_{n}^{Target}} {\vert}^{-}}{N}$$

wherein: RF=number of repertoire food types (i.e. food types normally consumed by the child); Q_{f}^{obs}=average amount of each repertoire food type; NRF=number of non-repertoire food types (i.e. food types not normally consumed by the child); Q_{f}^{average}=average amount of each non-repertoire food type; Q_{f}^{opt}=optimised amount of each food type; N=number of nutrients; Nutrientₙ^{Target}=nutritional target of nutrient n; Nutrientₙ^{opt}=optimised amount of nutrient n; and Wₙ=weight for nutrient n.

Wₙ may take any suitable positive values, depending on which nutrient is prioritised. For example, Wₙ may take a value of from 100 to 1000, optionally except for energy and sodium. In some embodiments, Wₙ=1000, except for energy and sodium.

### Mathematical Optimisation

The optimised complementary feeding diet may be determined by minimising the deviation or weighted sum using any suitable method known in the art. Suitable methods include any mathematical optimisation or mathematical programming method which can be used to minimise the deviation or weighted sum, such as convex programming or the like.

In some embodiments, linear programming is used to minimise the deviation or weighted sum.

“Linear programming” is a method for the optimisation of a linear objective function, subject to linear equality and linear inequality constraints. A Linear programming model may be defined by a list of variables (e.g. amount of each food type), a set of constraints (e.g. on nutrient and energy requirements and dietary diversity) and an objective function aiming to be optimised.

The “objective function” is the expression to be minimised or maximised. For example, in the present invention the objective function may be:
(i) the (negative) deviation between the nutrient targets (and optionally energy target) and the amount of each nutrient (and optionally energy) in the child's diet;
(ii) the deviation between the average intakes and the amount of each food type in the child's diet; or
(iii) the weighted sum of (i) the average (negative) deviation between the nutrient targets (and optionally energy target) and the amount of each nutrient (and optionally energy) in the child's diet and (ii) the average deviation between the average intakes and the amount of each food type in the child's diet.

In some embodiments, the objective function is the weighted sum of (i) the average (negative) deviation between the nutrient targets (and optionally energy target) and the amount of each nutrient (and optionally energy) in the child's diet and (ii) the average deviation between the average intakes and the amount of each food type in the child's diet.

In the present invention more than one objective function may be used. For example, in addition to the objective functions described herein, the method could comprise using one or more additional objective functions, such as cost of the complementary feeding diet. When more than objective function is used, the optimised complementary feeding diet may be determined by any multi-objective optimisation method known in the art.

In the present invention, the variables may be the amount of each food type. For example, the variables may be the amount of each of the following food types in the complementary feeding diet:
apples, banana, citrus, berries, other fruits (e.g. pear, peach, pineapple), and dried fruits;
orange/red vegetables (e.g. carrots), dark green vegetables (e.g. broccoli, spinach), starchy vegetables (e.g. sweet potato, peas), other vegetables (e.g. cauliflower, cabbage, green beans, zucchini), and avocado;
dried beans/peas/legumes, and nuts/seeds;
beef, chicken/turkey, eggs, lamb/goat/game, pork, fish/shellfish, and organ meats, optionally bacon/sausage/cold cuts;
infant cereal, hot cereal, white potato, and rice/pasta, optionally white bread/rolls/tortilla/bagel, optionally wholegrain bread/rolls/tortilla/bagel, optionally rice cakes/pretzels/crackers, optionally unsweetened ready-to-eat (RTE) cereal;
yogurt and cheese; and
butter, margarine, cream, and oil, optionally dressing, optionally mayonnaise.

In the present invention, the set of constraints may be any of the constraints described herein, such as the nutrient and energy requirements of the complementary feeding diet and constraints to ensure that the complementary feeding diet is diverse, age-appropriate, palatable, realistic, and/or generalisable. The set of constraints may be based on the sex of the child, the age of the child, and/or the weight of the child. Suitably, the set of constraints are based on the age of the child. For example:
(i) if the child is aged from 6 months to less than 9 months the complementary feeding diet may be constrained to provide: (1) about 195 kcal/day, (2) nutrient requirements based on the nutrient requirements for a child aged from 6 months to less than 9 months, and (3) all of the following criteria: (a) at least 10 g/day of two types of fruit; (b) at least 10 g/day of three types of vegetable; and (c) no more than 50 g/day egg and no more than 150 g/week egg; (d) no more than 21 g/day of any one type of meat;
(ii) if the child is aged from 9 months to less than 12 months the complementary feeding diet may be constrained to provide: (1) about 353 kcal/day; (2) nutrient requirements based on the nutrient requirements for a child aged from 9 months to less than 12 months, and (3) all of the following criteria: (a) at least 10 g/day of two types of fruit; (b) at least 10 g/day of three types of vegetable; (c) no more than 50 g/day egg and no more than 150 g/week egg; and (d) no more than 21 g/day of any one type of meat; or
(iii) if the child is aged from 12 months to less than 24 months the complementary feeding diet may be constrained to provide: (1) about 602 kcal/day, (2) nutrient requirements based on the nutrient requirements for a child aged from 9 months to less than 12 months, and (3) all of the following criteria: (a) at least 10 g/day of two types of fruit; (b) at least 10 g/day of three types of vegetable; (c) no more than 50 g/day egg and no more than 150 g/week egg; (d) no more than 21 g/day of any one type of meat; and (e) a limit of 10% total energy from occasional foods.

### Use of the Complementary Feeding Diet

In one aspect, the present invention provides for use of such a complementary feeding diet to meet all or substantially all the nutrient and energy requirements of a child during the complementary feeding period. The complementary feeding diet may be tailored to the child, based on the on the child's diet, the sex of the child, the age of the child, and/or the weight of the child.

In some embodiments, the present invention provides for use of a complementary feeding diet obtained or obtainable by the method of the invention to meet all or substantially all the nutrient and energy requirements of a child during the complementary feeding period, wherein the complementary feeding diet is determined based on the child's diet and the age of the child. For example, the complementary feeding diet may be determined based on: (i) whether the child is breast-fed, formula-fed, breast-fed and formula-fed, or milk-fed (e.g. cow's milk fed); (ii) whether the complementary feeding diet is fortified or unfortified; and/or (iii) whether the child is aged from 6 months to less than 9 months, from 9 months to less than 12 months, or from 12 months to less than 24 months.

Fortificant and/or Supplement Doses

The present invention also provides a method for determining fortificant and/or supplement doses, and fortificant and/or supplement doses obtained or obtainable by said method.

As used herein, a “fortificant” or “additive” is any suitable micronutrient which is added to food in the process of food fortification or enrichment. For example, a fortificant may be iron, folic acid, or other vitamins added to a wheat-based product or vitamins A and D added to cow's milk.

As used herein, a “supplement” or “dietary supplement” may be used to complement the nutrition of an individual (it is typically used as such but it might also be added to any kind of compositions intended to be ingested by the individual). The supplement may be in any form suitable for intake by a child and may comprise any suitable nutrients.

As described above, the present invention may be used to provide a complementary feeding diet to meet all the nutrient and/or energy requirements of the child's diet during the complementary feeding period. However, in some cases the complementary feeding diet only meets substantially all the nutrient and/or energy requirements of the child's diet during the complementary feeding period. For example, it may be challenging to obtain a complementary feeding diet which is diverse, age-appropriate, palatable, realistic and/or generalisable and which also meets the vitamin D requirements of the child's diet.

The present invention can be used to guide fortification or supplement plans. For example, when it is challenging to obtain a complementary feeding diet which is diverse, age-appropriate, palatable, realistic and/or generalisable and which also meets the requirements of the child's diet.

Suitably, when the complementary feeding diet does not meet one or more nutrient requirements of the child's diet during the complementary feeding period, suitable fortificant and/or supplement doses may be determined to meet said nutrient requirements. For example, fortificant and/or supplement doses may be determined by a method comprising: (a) determining a complementary feeding diet by any method described herein; and (b) determining fortificant and/or supplement doses required to meet the nutrient requirements of the child's diet during the complementary feeding period.

The skilled person will readily be able to determine suitable fortificant and/or supplement doses based on the nutrients provided by the complementary feeding diet and the one or more nutrient requirements which are not met by the complementary feeding diet. For example, if the complementary feeding diet provides about 2 mg/day vitamin D and the vitamin D requirements are 10 mg/day, the fortificant and/or supplement doses may be 8 mg/day vitamin D or more or about 8 mg/day vitamin D, e.g. the complementary food diet may be fortified with 8 mg/day vitamin D or more or about 8 mg/day vitamin D. For example, if the complementary feeding diet provides about 10 mg/day DHA and the DHA requirements are about 100 mg/day, the fortificant and/or supplement doses may be 90 mg/day DHA or more or about 90 mg/day DHA, e.g. the child's diet may include a supplement comprising 90 mg/day DHA or more or about 90 mg/day DHA.

The fortificant and/or supplement doses can be expressed in any suitable measurement unit. For example, in milligrams per day (mg/d) or micrograms per day (mcg/d).

In one aspect, the present invention provides for use of such a fortificant and/or supplement dose to supplement a child's diet during the complementary feeding period. The fortificant and/or supplement dose may be tailored to the child, based on the child's diet, the sex of the child, the age of the child, and/or the weight of the child.

In some embodiments, the present invention provides for use of a fortificant and/or supplement dose to supplement a child's diet during the complementary feeding period, wherein the dose is determined based on the child's diet and the age of the child. For example, the dose may be determined based on: (i) whether the child is breast-fed, formula-fed, breast-fed and formula-fed, or milk-fed (e.g. cow's milk fed); (ii) whether the complementary feeding diet is fortified or unfortified; and/or (iii) whether the child is aged from 6 months to less than 9 months, from 9 months to less than 12 months, or from 12 months to less than 24 months.

In some embodiments:
(a) the fortificant and/or supplement dose is a vitamin D fortificant and/or supplement dose and the child is aged from 6 months to less than 9 months, from 9 months to less than 12 months, or from 12 months to less than 24 months;
(b) the fortificant and/or supplement dose is a DHA fortificant and/or supplement dose and the child is aged from 12 months to less than 24 months; or
(c) the fortificant and/or supplement dose is for an iron fortificant and/or supplement dose and the child is from 6 months to less than 9 months, breast-fed, and optionally the complementary feeding diet is unfortified.

### Computer Program and Computer-Readable Medium

The methods described herein may be computer-implemented methods. For example, in one aspect, the present invention provides a computer-implemented method for determining a complementary feeding diet providing a plurality of food types to meet the nutrient and energy requirements of a child's diet during the complementary feeding period, comprising:
(a) providing (i) nutrient targets for a plurality of nutrients and (ii) average intakes for the plurality of food types; and
(b) determining the complementary feeding diet by minimising the weighted sum of (i) the average negative deviation between the nutrient targets and the amount of each nutrient in the child's diet and (ii) the average deviation between the average intakes and the amount of each food type in the child's diet.

In one aspect, the present invention provides a data processing system comprising means for determining a complementary feeding diet providing a plurality of food types to meet the nutrient and energy requirements of a child's diet during the complementary feeding period, given one or more objective functions, a set of variables, and a set constraints, as described herein.

In one aspect, the present invention provides a data processing apparatus comprising a processor configured to determine a complementary feeding diet providing a plurality of food types to meet the nutrient and energy requirements of a child's diet during the complementary feeding period, given one or more objective functions, a set of variables, and a set constraints, as described herein.

In one aspect, the present invention provides a computer program comprising instructions which, when the program is executed by a computer, cause the computer to determine a complementary feeding diet providing a plurality of food types to meet the nutrient and energy requirements of a child's diet during the complementary feeding period, given one or more objective functions, a set of variables, and a set constraints, as described herein.

In one aspect, the present invention provides a computer-readable medium comprising instructions which, when executed by a computer, cause the computer to determine a complementary feeding diet providing a plurality of food types to meet the nutrient and energy requirements of a child's diet during the complementary feeding period, given one or more objective functions, a set of variables, and a set constraints, as described herein.

The complementary feeding diet may be determined by any method described herein.

In one aspect, the present invention provides a data processing system comprising means for carrying out the method of the invention.

In one aspect, the present invention provides a data processing apparatus comprising a processor configured to perform the method of the invention.

In one aspect, the present invention provides a computer program comprising instructions which, when the program is executed by a computer, cause the computer to carry out the method of the invention.

In one aspect, the present invention provides a computer-readable medium comprising instructions which, when executed by a computer, cause the computer to carry out the method of the invention.

In one aspect, the present invention provides a computer-readable data carrier having stored thereon the computer program of the invention.

In one aspect, the present invention provides a data carrier signal carrying the computer program of the invention.

### EXAMPLES

The invention will now be further described by way of examples, which are meant to serve to assist the skilled person in carrying out the invention and are not intended in any way to limit the scope of the invention.

### Example 1—Diet Modelling to Optimise Complementary Diets

Dietary modelling using linear programming was applied to a Feeding Infants and Toddlers Study (FITS) data set to develop theoretical diets that satisfy nutrient requirements, while meeting the median estimated energy requirement (EER), for 6 to 8.9 month old and 9 to 11.9 month old infant with three possible milk sources: breast milk, formula, and a mixture of both. Whole cow's milk was the milk source considered for toddlers from 12 to 23.9 months.

The model can be used for any age group. The age groups of concern defined for the project were 6 to 8.9 months, 9 to 11.9 months, and 12 to 23.9 months to align with WHO guidance on complementary feeding.

The model can be used for any food supply. The models were run with US food supply, which is heavily fortified. For example iron, folic acid and other B vitamins are added to wheat, rice and maize and vitamins A and D are added to cow's milk. Then all runs were repeated without fortification to simulate the food supply in European countries such as France or Germany where fortification of grains and dairy products is not done.

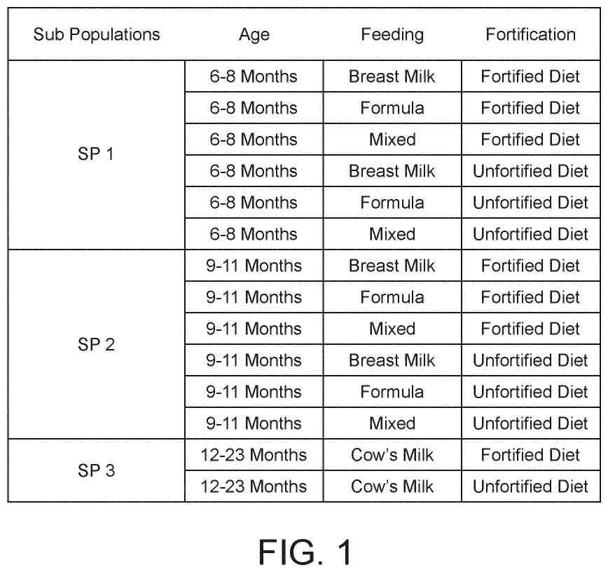

In total, there were 14 possible scenarios, resulting in 14 diet optimisations as shown in FIG. 1.

### Methods

### Estimated Energy Requirements

The model can be used to meet any energy requirement. Estimated Energy Requirements (EER) were calculated by applying the mathematical model defined by Butte N F Public health nutrition. 2005; 8(7a):953-67 according to the most recent WHO growth charts.

The 50th percentile for boys and girls (averaged) was used for the median EER for each age group. The median EER by age group was: 6 to 8.9 months: 636 kcals; 9 to 11.9 months: 706 kcals; and 12 to 23.9 months: 860 kcals.

### Milk Versus Complementary Food

The model can be used for any combination of milk and complementary food. The ratio of milk to complementary food in the diet was based on published observations (Butte N, et al. Journal of the American Dietetic Association. 2004; 104(3):442-54). The data for infants and toddlers was based on the average of several observations in industrialised and developing countries. The average proportion of energy from complementary foods by age group was: 6 to 8.9 months: 30%; 9 to 11.9 months: 50%; and 12 to 23.9 months: 70%.

The volume of milk was calculated based on the proportion of energy from milk versus complementary food and the EER per day, according to age. The energy contribution from milk per day was fixed in the model.

For mixed-fed infants, the proportion of breast milk to formula in the diet was fixed at 50:50 for the optimisation models. Analyses of the FITS data indicated that the actual ratio in the observed diets of mixed fed infants was 46:54 for 6 to 8.9 month olds and 50:50 for 9 to 11.9 month olds. For toddlers, ages 12 to 23.9 months old, it was assumed that they are consuming whole cow's milk. Since this is the milk recommended for this age group by many authoritative groups (Agostoni C, et al. Journal of pediatric gastroenterology and nutrition. 2008; 46(1):99-110).

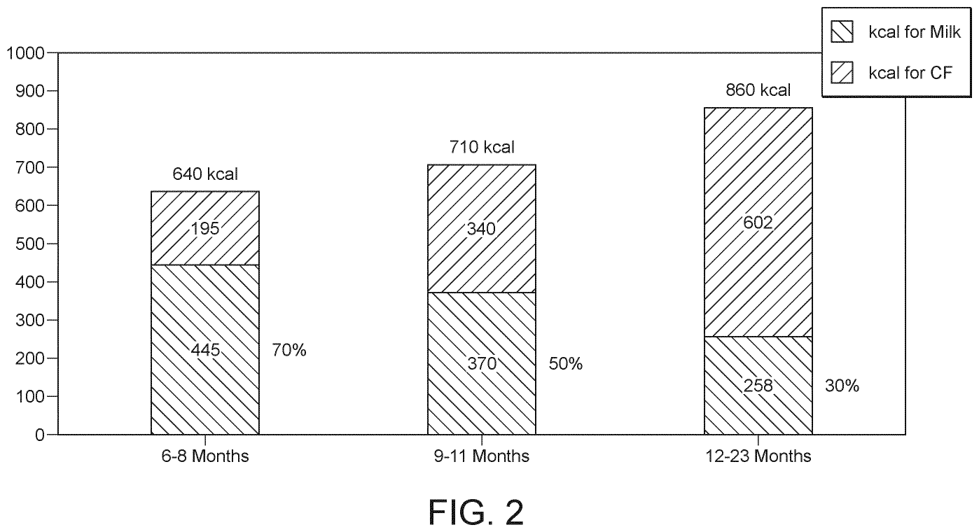

The estimated energy available for complementary food by age group can be seen in FIG. 2.

### Nutrient Compositions of Milks

The model can be used for any source of milk. Mature breast milk composition was based on averaged data from several different countries as breast milk composition differs according to multiple variables, particularly in terms of fatty acid profile and vitamin content. A global composition of infant formula (FUF) for infants ages 6 to 12 months was calculated with an average of 7 products from various countries (see Table 1).

TABLE 1
Exemplary milk compositions: global average mature breast
milk (BM) and formula (FUF) consumed by infants

| | | Global average | |
|---|---|---|---|
| | | mature breast | Global average |
| Nutrient | Unit | milk (per 100 ml) | FUF (per 100 ml) |
| Energy | kcal | 65 | 66 |
| Protein | g | 1.0 | 1.80 |
| Total lipid | g | 3.5 | 3.10 |
| Carbohydrate | g | 6.8 | 8.07 |
| SFA | g | 1.5 | 1.20 |
| MUFA | g | 1.3 | 1.00 |
| PUFA | g | 0.5 | 0.60 |
| ALA | mg | 34.5 | 64.80 |
| DHA | mg | 7.0 | 9.60 |
| Available carb | g | 6.9 | 8.07 |
| Sodium | mg | 14.3 | 22.60 |
| Potassium | mg | 46.7 | 80.10 |
| Calcium | mg | 27.4 | 66.90 |
| Iron | mg | 0.07 | 0.90 |
| Zinc | mg | 0.17 | 0.60 |
| Vitamin A | μg | 58.3 | 62.90 |
| Vitamin D | μg | 0.1 | 1.20 |
| Vitamin C | mg | 6.5 | 8.90 |
| Folate (DFE) | μg | 7.9 | 14.29 |
| Vitamin B6 | μg | 13.25 | 60.00 |
| Vitamin B12 | μg | 0.06 | 0.13 |
| Vitamin E | mg | 0.10 | 1.09 |
| Magnesium | mg | 3.13 | 5.42 |
| Phosphorus | mg | 13.42 | 42.05 |

The calculated averages for energy and macronutrients agree with data based on a meta-analysis (Hester S N, et al Journal of nutrition and metabolism. 2012; Article ID 891201) and a systematic review (Gidrewicz D A, Fenton T R BMC pediatrics. 2014; 14(1):216).

The composition of whole milk cow's milk (both fortified and unfortified) was taken from the USDA database. All Nutrient data for both milks was from SR 01078 (USDA: Nutrient data for: Milk, whole, 3.7% milk fat, without added vitamin A and vitamin 0) and fortified levels of vitamins A and 0 was from SR 01211 (see Table 2).

TABLE 2
Exemplary milk compositions: whole milk consumed by toddlers

| | | Unfortified cow's | Fortified cow's |
|---|---|---|---|
| Nutrient | Unit | milk (per 100 ml) | milk (per 100 ml) |
| Energy | kcal | 62.14 | 62.14 |
| Protein | g | 3.18 | 3.18 |
| Total lipid | g | 3.55 | 3.55 |
| Carbohydrate | g | 4.51 | 4.51 |
| SFA | g | 2.21 | 2.21 |
| MUFA | g | 1.03 | 1.03 |
| PUFA | g | 0.13 | 0.13 |
| ALA | mg | 0.05 | 0.05 |
| DHA | mg | 0.00 | 0.00 |
| Available carb | g | 4.51 | 4.51 |
| Sodium | mg | 47.57 | 47.57 |
| Potassium | mg | 146.60 | 146.60 |
| Calcium | mg | 115.53 | 115.53 |
| Iron | mg | 0.05 | 0.05 |
| Zinc | mg | 0.36 | 0.36 |
| Vitamin A | μg | 19.42 | 57.28 |
| Vitamin D | μg | 0.05 | 1.19 |
| Vitamin C | mg | 1.46 | 1.46 |
| Folate (DFE) | μg | 4.85 | 4.85 |
| Vitamin B6 | μg | 0.04 | 0.04 |
| Vitamin B12 | μg | 0.35 | 0.35 |
| Vitamin E | mg | 0.00 | 0.00 |
| Magnesium | mg | 12.62 | 12.62 |
| Phosphorus | mg | 90.29 | 90.29 |

### Food Database Preparation

For the model, any suitable foods can be used. The composition of foods was from the Nutrition Data System for Research (NDSR, version 2015: University of Minnesota, Minneapolis, MN).

The NDSR contains foods with fortification unlike the equivalent foods from other countries. Product compositions from the grains and the dairy group were manually checked for fortification. For such foods, the macronutrient composition from the NDSR was still applied but the micronutrient composition of unfortified foods, from Ciqual (CIQUAL food composition table 2017), was used.

The food database was cleaned by removing:
Foods with no or low nutritional contribution (e.g. condiments)
Developmentally and age inappropriate foods: Some foods were considered developmentally inappropriate for infants <12 mo. For toddlers (12-23.9 mo.) most foods kept as long as they could be prepared in an appropriate way e.g. raw carrots could be grated. Guidance on the developmental appropriateness of foods was sought from the Encyclopedia of Food Safety (Altkorn G R, et al. Encyclopedia of Food Safety. 2014). Only unsweetened RTE breakfast cereals were included as many RTE breakfast cereals are high in sugar and would likely not have a place in optimised infant/toddler diets.
Dietary supplements and baby foods (e.g. fruit purees, baby food dinners etc., excluding infant cereal).
Mixtures: Mixed dishes were eliminated since they consist of more than one food group e.g. pizza consists of grains and vegetable and dairy. Mixed vegetables and mixed fruits were eliminated too.
Occasional foods such as sweets, savory snacks, sugar sweetened beverages and 100% juices were removed completely. However, for toddlers a 10% energy allowance was left for this category such that milk and the core food groups had to provide 90% of the EER.
Foods for special diets (e.g. plant based milks, egg substitutes, vegetarian meats).
Region-specific foods (e.g. pancakes, French toast, biscuits, peanut butter).

Although greater than 4000 individual food items were originally present in the database of foods consumed by the population concerned (6-23.9 months), the elimination of foods per age group resulted in: 228 individual foods for 6-8.9 months; 419 individual foods for 9-11.9 months; 1129 individual foods for 12-23.9 months.

For the model, the foods may be grouped in any suitable way. The foods were grouped into major and minor food groups as shown in FIG. 3. For example, a major food group would be the meat group and the minor food groups therein would include beef, chicken/turkey, eggs. For infants, yogurt and cheese were considered as contributing to the complementary food part of the diet, rather than the milk part, since neither cheese nor yogurt can replace breast milk or formula. However, for toddlers, energy from yogurt/cheese is assigned to milk or dairy since the former can replace cow's milk. The nutrient composition of foods was based on the average nutritional composition of the products in a given minor food group.

### Nutrient Requirements and Targets

The model can be used to meet any nutrient requirements and with any nutrient targets. The macronutrient targets are mainly from WHO/FAO with the exception of the maximum limit for protein, which is based on ESPGHAN recommendations. The WHO definition and recommendation on free sugars were applied and free sugars were estimated by adding the total sugars in fruit juice to the added sugars values for each food sub group.

The micronutrient targets are mainly from WHO/FAO with the exception of sodium, potassium, magnesium, vitamin B12, vitamin D and dietary fiber, which are based on other published recommendations.

Absorbed iron rather than total iron was included to reflect the differences between breast milk iron compared with the iron in infant formula and the difference between haem versus non-haem iron. The quantities of absorbed iron per food sub group were estimated using published values and are shown in Table 3 below.

TABLE 3
Percentage iron absorption according to the dietary source of iron

| Iron source | % absorption |
|---|---|
| Breast milk | 50% |
| Follow up formula | 15-20% |
| Fortified foods/supplements/non | 10% |
| heam iron sources | |
| Haem iron | 25% |

An exemplary list of nutrient targets, minima, and maxima for each group are shown in FIG. 4.

### Linear Programming Model

Fourteen Linear Programming models were run depending on age, breast feeding status and fortification of the food supply (see FIG. 1). One Linear programming model is defined by a list of variables, a set of constraints, and an objective function aiming to be optimised.

The variables used in the models were the amount of the 38 food subgroups. The optimised amounts are obtained by satisfying all the constraints of the model and by optimising the objective function. Any suitable food groups could be used.

In each model, any suitable constraints may be used. A set of nutritional constraints was defined in order to fix the possible range of each nutrient, including energy. A set of constraints was also used to limit the amount of each food subgroup and each food group. The maximum amount was estimated, separately for each age group, as the 95th percentile of the distribution among FITS intakes. A set of food diversity constraints was also added (see next section).

Any suitable objective function could be used. The objective function in the model was based on a multi-criteria approach:
1. To minimise the average deviation between the average observed amount and optimised amount for each food subgroup.
2. To minimise the average negative deviation for all individual nutrients between the nutritional target and the nutrient content in the optimised diet. The negative deviation was taken into account to estimate the missing amount of nutrient to reach the targets

The objective function for the model was as follows (for each feeding group):

$$\frac{{\sum }_{f = 1}^{f = RF} \vert \frac{{Q}_{f}^{opt} - {Q}_{f}^{obs}}{{Q}_{f}^{obs}} \vert + {\sum }_{f = 1}^{f = NRF} \vert \frac{{Q}_{f}^{opt}}{{Q}_{f}^{average}} \vert}{R F + N R F} + \frac{{\sum }_{n = 1}^{n = N} {w}_{n} \vert \frac{{nutrient}_{n}^{opt} - {Nutrient}_{n}^{Target}}{{Nutrient}_{n}^{Target}} {\vert}^{-}}{N}$$

### Where:

RF=number of repertoire food minor groups (i.e. as consumed in the corresponding feeding group) for a given feeding group (e.g. breast-fed consumers)
Q_{f}^{obs}=average amount of repertoire food subgroups consumed in the corresponding feeding group.
NRF=number of non-repertoire food subgroups (i.e. food subgroups non-consumed in the corresponding feeding group but consumed in another feeding group within the same subpopulation)
Q_{f}^{average}=average amount of non-repertoire food subgroups consumed in the other feeding groups within the same subgroup
Q_{f}^{opt}=optimised amount of food subgroups
N=number of nutrients
Nutrientₙ^{Target}=nutritional target of nutrient n
Nutrientₙ^{opt}=optimised amount of nutrient n
Wₙ=weight for nutrient n

This model expresses:
1. the deviation to food items in percentage, which favours the dietary shifts of food items already consumed in high quantities.
2. the nutritional negative deviation from nutritional target in percentage, which favours the nutritional targets being achieved.

Any suitable weighted sum can be used. In the model, the nutritional targets were defined with a higher priority versus average intakes indicating that a greater deviation from intake could be accepted to facilitate the achievement of nutritional targets. All weights are set to 1000 except for energy and sodium to help reach the nutritional targets.

The models can be run for any time scale. The models were run as an average day in a 7 day week, so that the results could be translated into 7 day menus. Menus spanning 7 days were considered appropriate since nutrient references are typically intended to be achieved within 1 week.

### Dietary Diversity Constraints

Using a 7-day approach, certain constraints were introduced to minimise monotonous diets, increase the variety within food groups, and improve dietary diversity, which is especially important during complementary feeding. Exposure to a wide variety of flavours during early feeding has been reported to increase acceptability at subsequent ages.

Any suitable constraints can be used. The following constraints were introduced in the model:
Fruits and vegetables: at least 2 fruits and 3 vegetable minor groups per day and at least 10 g for each food subgroup were added as a constraint to help ensure a variety of fruit and vegetables is recommended. Complementary feeding is a unique timeframe to offer a variety of fruits and vegetables to increase the likelihood of consuming the same in subsequent years.
Eggs: No more than one egg (50 g)/day and egg no more than 3 days/week. When we translate the daily menu into a 7 day week (50*3/7) this is equivalent to 21 g of egg on an average day.
Meat group: Similar to eggs, no more than 21 g/day was fixed for the maximum amount of any individual meat subgroup to help ensure a variety of meats are recommended.
Sweets, desserts, savory snacks & 100% juice: A limit of 10% total energy for toddlers was reserved for occasional foods, but no energy allowance for occasional foods was made in infant diets due to their low calorie allowance from complementary foods.

Any additional constraints may also be introduced. For example, all optimised diets for a given age group were restrained to meet the energy target i.e. optimised diets were isocaloric to reduce the variation between diets for a given age group.

### Results

### 6 to 8.9 Month Old Infants

The major food groups in the optimised diets are shown in FIGS. 5A-C in terms of quantity and energy contribution.

The quantities of food in the optimised diets were compared with the quantities in the observed diets and were found to be 10% to 30% less for the optimised mixed fed and formula fed, respectively. All macronutrient requirements and most micronutrient requirements were fulfilled (see FIG. 5D). Notably, the zinc requirement was fulfilled in all scenarios including the breast-fed unfortified diet. The DHA target was achieved in all scenarios too due to the contributions from breast milk and/or FUF. Magnesium was achieved in all scenarios. The free sugars content of the diets was negligible. The sodium was reasonably low and was coming from natural sources such as the intrinsic sodium in ‘other dairy products’.

### 9 to 11.9 Month Old Infants

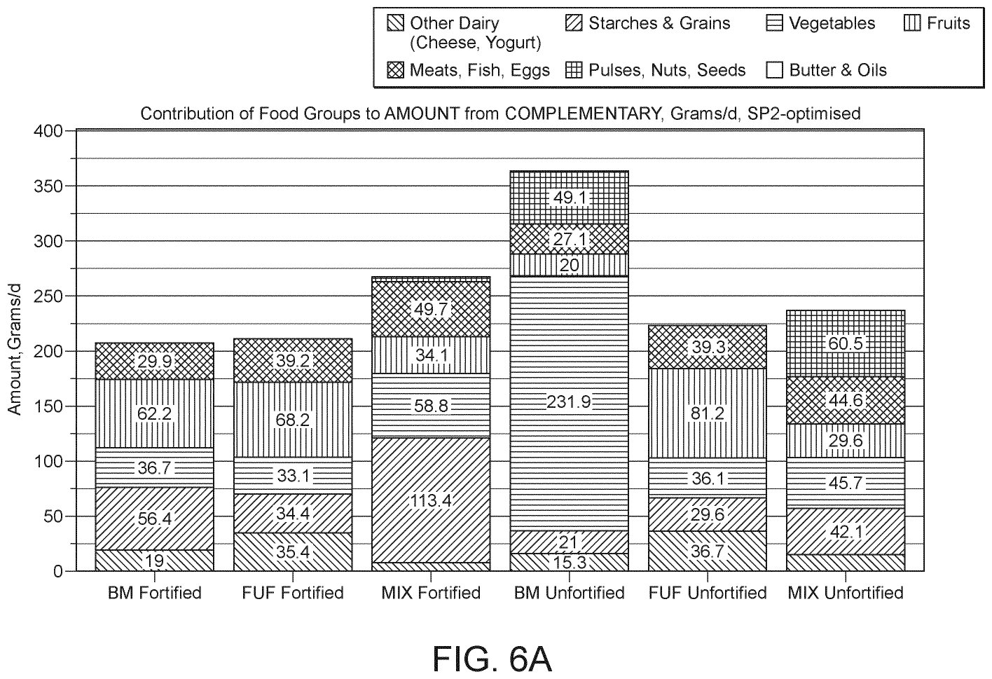

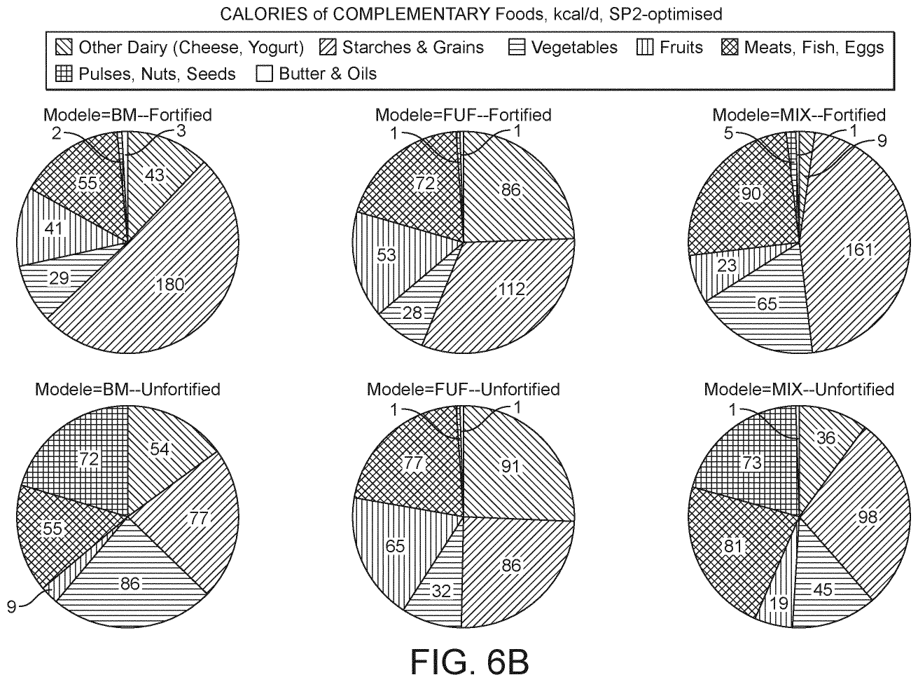

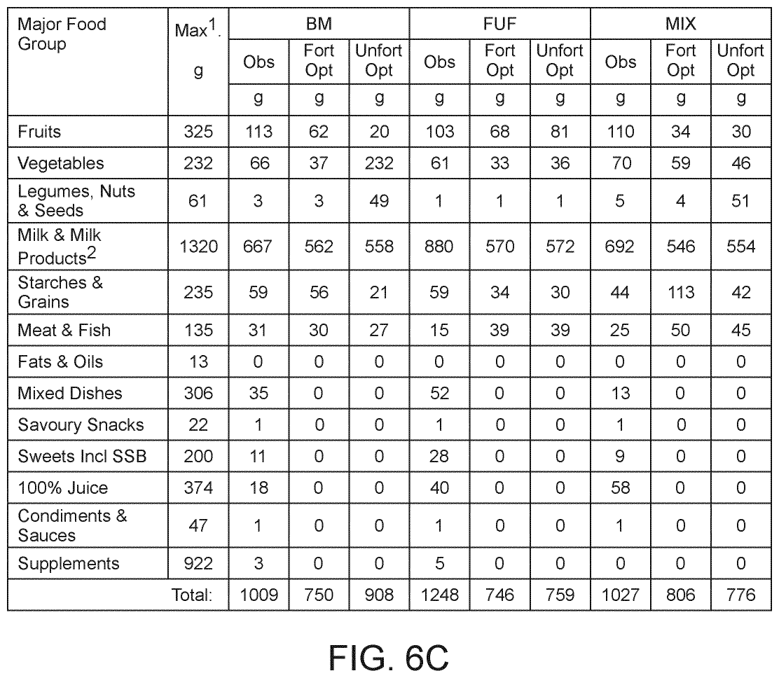

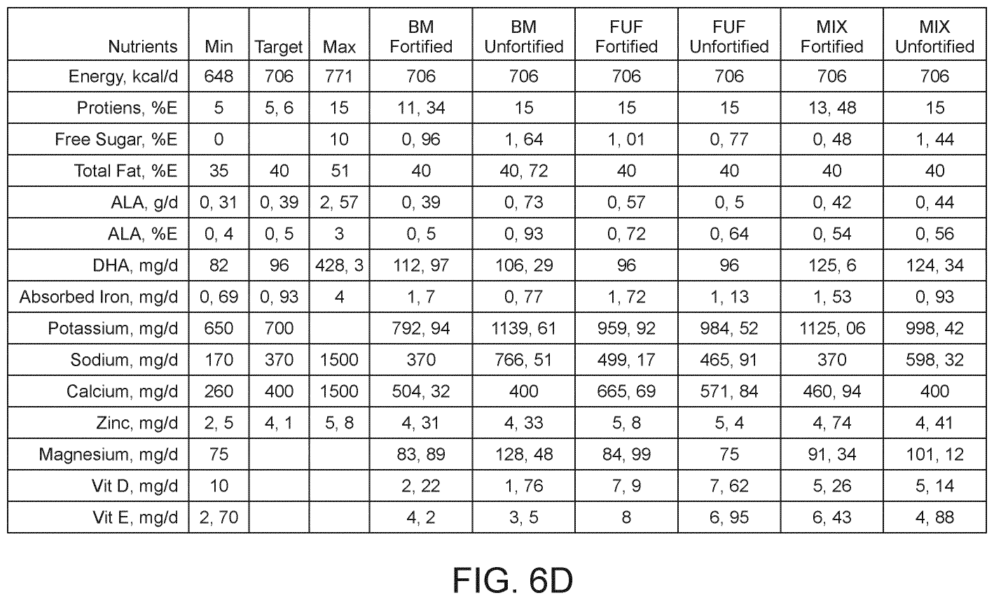

The major food groups in the optimised diets are shown in FIGS. 6A-C in terms of quantity and energy contribution.

The total amount of food and milk in the observed diets was on average 27% greater than the quantities in the optimised diets. All macronutrient requirements and most micronutrient requirements were fulfilled (see FIG. 6D). Notably, the absorbed iron was achieved in all subpopulations. The free sugars were negligible. The sodium was around the Al of 370 mg, except in the breast-fed unfortified diet due to the natural sodium intrinsically present in vegetables.

### 12 to 23.9 Month Old Toddlers

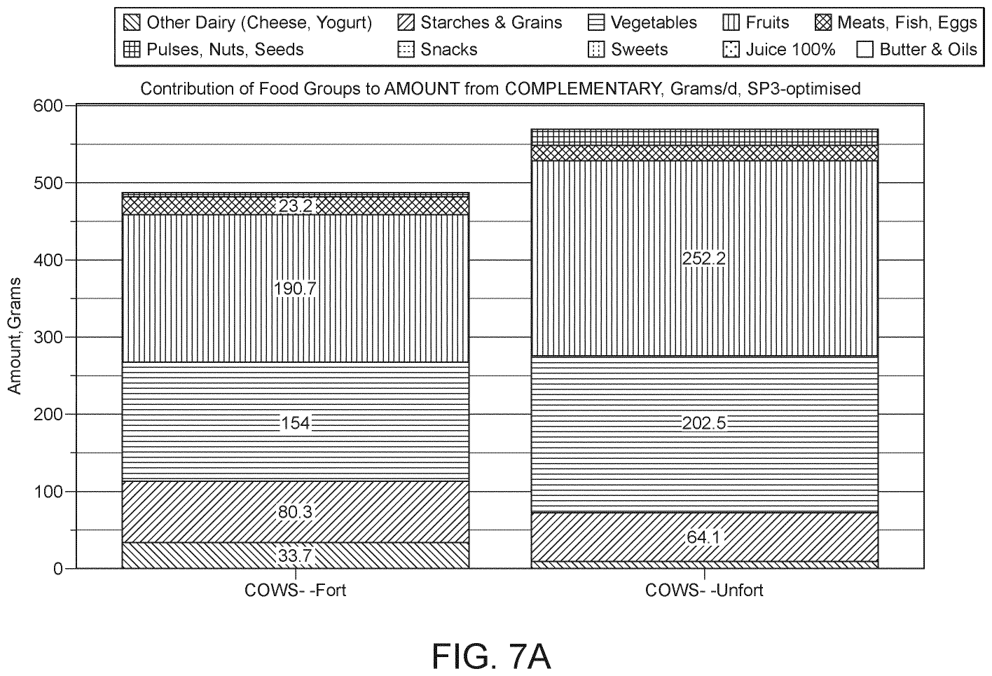

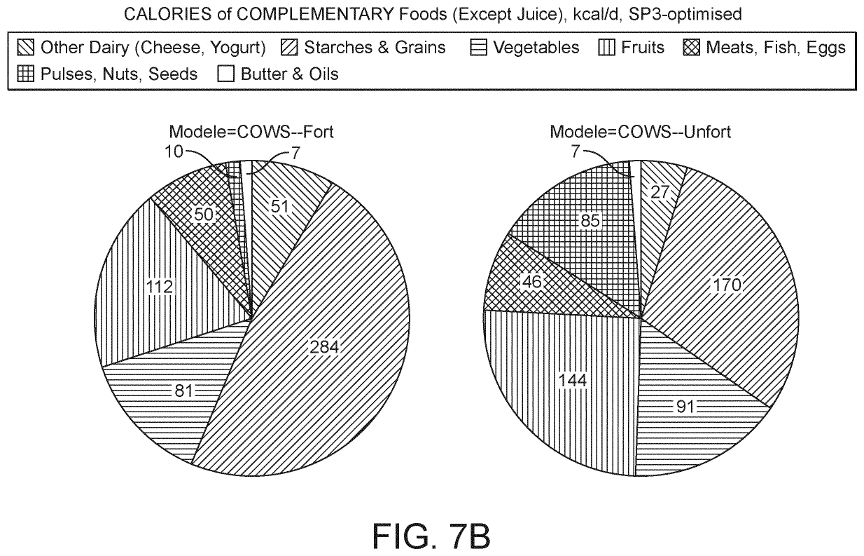

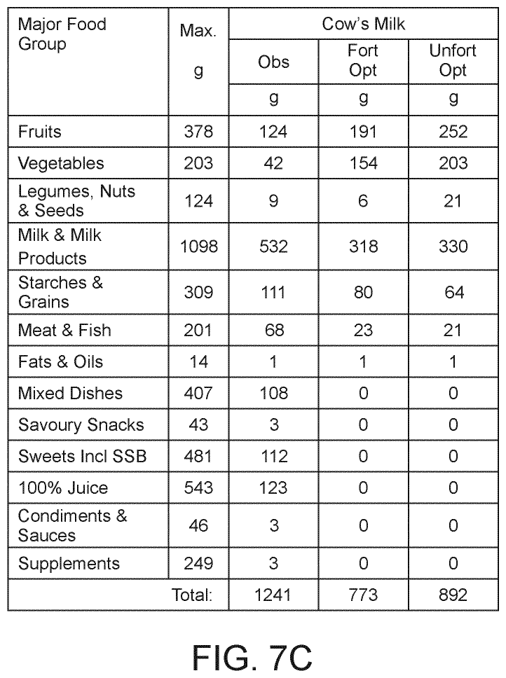

The major food groups in the optimised diets are shown in FIGS. 7A-C in terms of quantity and energy contribution.

The quantity of food, including milk, in the optimised diets is 33% less than the observed diets. The optimised diets for toddlers 12 to 23.9 months old met or exceeded most nutrient targets (see FIG. 7D). The sodium levels of these diets were close to the target. The free sugars content was well below the maximum limit of 10% of total energy.

### Conclusion

The optimised diets meet most nutrient requirements of the infants and toddlers and, in general, much less total food was needed to fulfil nutrient adequacy in all age groups compared with the observed intakes. The optimised diets differ with regard to the quantity of food group intakes, depending on the milk source (breast milk or formula) and the fortification of the food supply (US or European).

The optimised diets can be adapted as necessary. For example, the optimised diets could be adapted to include commercial baby foods or any other food types which were omitted. The optimised diets could also be adapted according to the energy requirements (EER) of the child, for example depending on the sex of the child or the child's weight.

The results can be used to guide fortification and/or supplementation. For example, fortification or supplementation can be used to provide adequate iron to breast-fed infants, particularly in the early months of complementary feeding, and vitamin D supplementation for infants and toddlers.

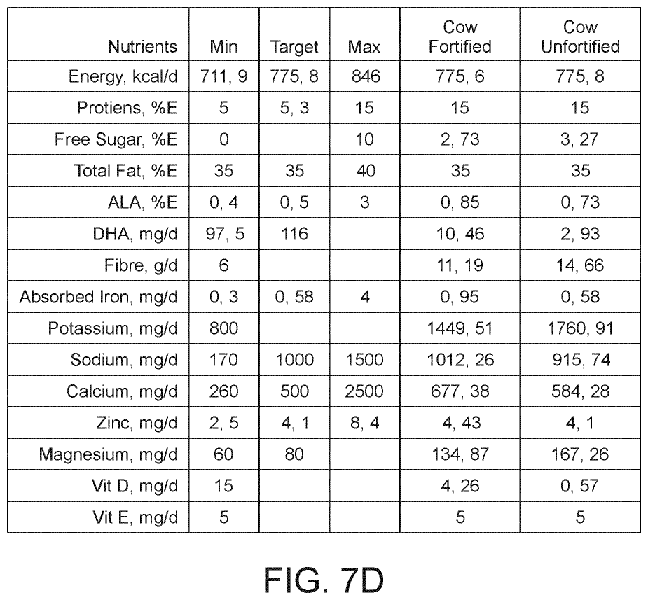

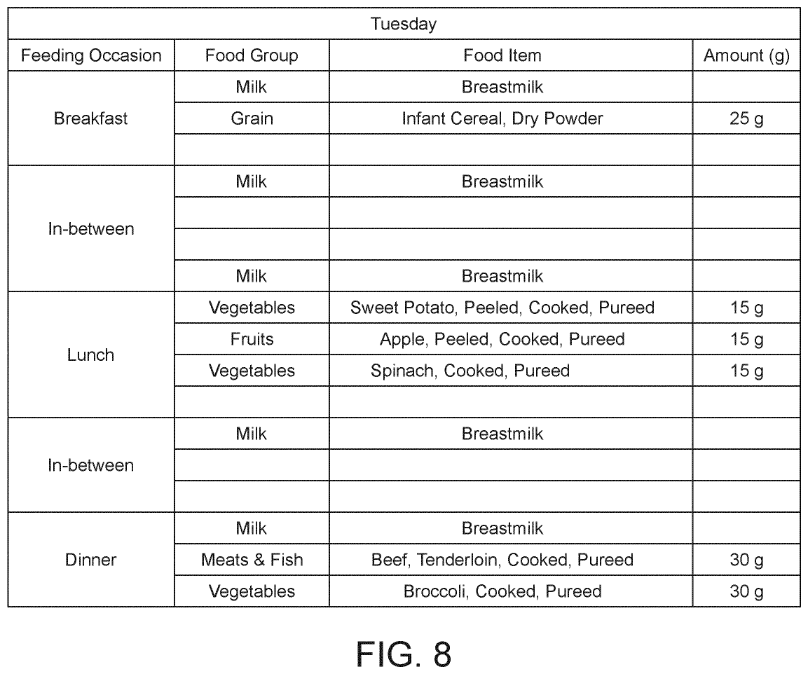

These results have been transferred into realistic weekly menu plans for infants and toddlers. A sample daily menu plan is shown in FIG. 8.

### EMBODIMENTS

Various preferred features and embodiments of the present invention will now be described with reference to the following numbered paragraphs (paras).

1. A method for determining a complementary feeding diet providing a plurality of food types to meet the nutrient and energy requirements of a child's diet during the complementary feeding period, comprising:
(a) providing (i) nutrient targets for a plurality of nutrients and (ii) average intakes for the plurality of food types; and
(b) determining the complementary feeding diet by minimising the weighted sum of (i) the average negative deviation between the nutrient targets and the amount of each nutrient in the child's diet and (ii) the average deviation between the average intakes and the amount of each food type in the child's diet.

2. A method for determining fortificant and/or supplement doses to meet the nutrient requirements of a child's diet during the complementary feeding period, comprising:
(a) providing (i) nutrient targets for a plurality of nutrients and (ii) average intakes for the plurality of food types;
(b) determining a complementary feeding diet providing a plurality of food types by minimising the weighted sum of (i) the average negative deviation between the nutrient targets and the amount of each nutrient in the child's diet and (ii) the average deviation between the average intakes and the amount of each food type in the child's diet; and
(c) determining fortificant and/or supplement doses required to meet the nutrient requirements of the child's diet during the complementary feeding period.

3. The method according to para 1 or 2, wherein the child is aged from 6 months to less than 9 months, from 9 months to less than 12 months, or from 12 months to less than 24 months.

4. The method according to any preceding para, wherein the child's diet comprises the complementary feeding diet and one or more of breast milk, infant formula, and cow's milk.

5. The method according to any preceding para, wherein the child's diet comprises the complementary feeding diet and breast milk; the complementary feeding diet and infant formula; the complementary feeding diet, breast milk and infant formula; or the complementary feeding diet and cow's milk.

6. The method according to any preceding para, wherein:
(i) the child is aged from 6 months to less than 9 months and the child's diet comprises the complementary feeding diet and breast milk;
(ii) the child is aged from 6 months to less than 9 months and the child's diet comprises the complementary feeding diet and infant formula;
(iii) the child is aged from 6 months to less than 9 months and the child's diet comprises the complementary feeding diet, breast milk and infant formula;
(iv) the child is aged from 9 months to less than 12 months and the child's diet comprises the complementary feeding diet and breast milk;
(v) the child is aged from 9 months to less than 12 months and the child's diet comprises the complementary feeding diet and infant formula;
(vi) the child is aged from 9 months to less than 12 months and the child's diet comprises the complementary feeding diet, breast milk and infant formula; or
(vii) the child is aged from 12 months to less than 24 months and the child's diet comprises the complementary feeding diet and cow's milk.

7. The method according to any one of paras 4 to 6, wherein the child's diet further comprises one or more supplements.

8. The method according to any preceding para, wherein the energy requirements of a child's diet are determined based on the weight of the child, preferably wherein the weight of the child is determined based on body weight values from 50th percentile of the Growth Chart Percentiles from the World Health Organization (WHO).

9. The method according to any preceding para, wherein the child's diet is isocaloric, preferably wherein:
(i) the child is aged from 6 months to less than 9 months and the child's diet comprises about 636 kcal/day;
(ii) the child is aged from 9 to less than 12 months and the child's diet comprises about 706 kcal/day; or
(iii) the child is aged from 12 months to less than 24 months and the child's diet comprises about 860 kcal/day.

10. The method according to any preceding para, wherein the complementary feeding diet is fortified or unfortified.

11. The method according to any preceding para, wherein the complementary feeding diet is diverse.

12. The method according to any preceding para, wherein the complementary feeding diet meets at least the following criteria:
(a) at least 10 g/day of two types of fruit;
(b) at least 10 g/day of three types of vegetable;
(c) no more than 50 g/day egg and no more than 150 g/week egg;
(d) no more than 21 g/day of any one type of meat; and
(e) optionally, a limit of 10% total energy from occasional foods.

13. The method according to any preceding para, wherein the plurality of food types comprises at least: one or more types of fruit; one or more types of vegetable; one or more types of pulse, nut or seed; one or more types of meat; one or more types of starch or grain; one or more type of dairy; and one or more type of oil and fat.

14. The method according to any preceding para, wherein the plurality of food types excludes one or more food types, optionally wherein the one or more excluded food types comprise one or more of:
(a) foods with low or no nutrient contribution;
(b) developmentally or age inappropriate foods;
(c) occasional foods, for example 100% fruit juice;
(d) supplements;
(e) foods for special diets;
(f) regional-specific foods; and
(g) mixed foods.

15. The method according to any preceding para, wherein the complementary feeding diet is determined using linear programming.

16. The method according to any preceding para, wherein the nutrient targets comprise targets for at least protein, lipids, iron, calcium, and zinc.

17. The method according to para 16, wherein the nutrient targets further comprise targets for ALA and/or DHA.

18. The method according to para 16 or 17, wherein the nutrient targets further comprise targets for one or more of magnesium, phosphorus, sodium, and potassium.

19. The method according to any one of paras 16 to 18, wherein the wherein the nutrient targets further comprise targets for vitamin A, vitamin C, and folate.

20. The method according to any one of paras 16 to 19, wherein the nutrient targets further comprise targets for one or more of vitamin D, vitamin B6, vitamin B12, vitamin E.

21. The method according to any preceding para, wherein the nutrient targets take into account bioavailability for one or more nutrient, preferably the target for iron takes into account bioavailability of iron.

22. The method according to any preceding para, wherein the weighted sum gives a greater weighting to (i) the average negative deviation between the nutrient targets and the amount of each nutrient in the child's diet.

23. A computer program comprising instructions which, when the program is executed by a computer, cause the computer to carry out the method of any one of paras 1 to 22.

24. A computer-readable data carrier having stored thereon the computer program of para 23.

25. A computer-readable medium comprising instructions which, when executed by a computer, cause the computer to carry out the method of any one of paras 1 to 22.

26. A complementary feeding diet obtained by the method of any one of paras 1 to 22.

27. Use of a complementary feeding diet according to para 26 to meet the nutrient and energy requirements of a child during the complementary feeding period.

28. A fortificant and/or supplement dose obtained by the method of any one of paras 2 to 22.

29. Use of a fortificant and/or supplement dose according to para 28 to supplement a child's diet during the complementary feeding period.

30. Use according to para 29, wherein:
(a) the fortificant and/or supplement dose is a vitamin D fortificant and/or supplement dose and the child is aged from 6 months to less than 9 months, from 9 months to less than 12 months, or from 12 months to less than 24 months;
(b) the fortificant and/or supplement dose is a DHA fortificant and/or supplement dose and the child is aged from 12 months to less than 24 months; or
(c) the fortificant and/or supplement dose is for an iron fortificant and/or supplement dose and the child is from 6 months to less than 9 months and is breast-fed.

All publications mentioned in the above specification are herein incorporated by reference. Various modifications and variations of the disclosed methods, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A method for determining a complementary feeding diet providing a plurality of food types to meet the nutrient and energy requirements of a child's diet during the complementary feeding period, comprising:
(a) providing (i) nutrient targets for a plurality of nutrients and (ii) average intakes for the plurality of food types; and
(b) determining the complementary feeding diet by minimising the weighted sum of (i) the average negative deviation between the nutrient targets and the amount of each nutrient in the child's diet and (ii) the average deviation between the average intakes and the amount of each food type in the child's diet.

2. A method for determining fortificant and/or supplement doses to meet the nutrient requirements of a child's diet during the complementary feeding period, comprising:
(a) providing (i) nutrient targets for a plurality of nutrients and (ii) average intakes for the plurality of food types;
(b) determining a complementary feeding diet providing a plurality of food types by minimising the weighted sum of (i) the average negative deviation between the nutrient targets and the amount of each nutrient in the child's diet and (ii) the average deviation between the average intakes and the amount of each food type in the child's diet; and
(c) determining fortificant and/or supplement doses required to meet the nutrient requirements of the child's diet during the complementary feeding period.

3. The method according to claim 1, wherein the child is aged from 6 months to less than 24 months.

4. The method according to claim 1, wherein the child's diet comprises the complementary feeding diet and one or more of breast milk, infant formula, and cow's milk.

5. The method according to claim 1, wherein the complementary feeding diet is fortified or unfortified.

6. The method according to claim 1, wherein the complementary feeding diet is diverse, and wherein the complementary feeding diet meets a criteria selected from the group consisting of:
(a) at least 10 g/day of two types of fruit;
(b) at least 10 g/day of three types of vegetable;
(c) no more than 50 g/day egg and no more than 150 g/week egg;
(d) no more than 21 g/day of any one type of meat; and
(e) a limit of 1000 total energy from occasional foods.

7. The method according to claim 1, wherein the plurality of food types comprises at least food type selected from the group consisting of: fruit; vegetable; pulse, nut or seed; meat; starch or grain; dairy; and oil and fat.

8. The method according to claim 1, wherein the nutrient targets comprise targets for at least protein, lipids, iron, calcium, and zinc.

9-15. (canceled)

16. The method according to claim 2, wherein the child is aged from 6 months to less than 24 months.

17. The method according to claim 2, wherein the child's diet comprises the complementary feeding diet and one or more of breast milk, infant formula, and cow's milk.

18. The method according to claim 2, wherein the complementary feeding diet is fortified or unfortified.

19. The method according to claim 2, wherein the complementary feeding diet is diverse, and wherein the complementary feeding diet meets a criteria selected from the group consisting of:
(a) at least 10 g/day of two types of fruit;
(b) at least 10 g/day of three types of vegetable;
(c) no more than 50 g/day egg and no more than 150 g/week egg;
(d) no more than 21 g/day of any one type of meat; and
(e) a limit of 10% total energy from occasional foods.

20. The method according to claim 2, wherein the plurality of food types comprises at least food type selected from the group consisting of: fruit; vegetable; pulse, nut or seed; meat; starch or grain; dairy; and oil and fat.

21. The method according to claim 2, wherein the nutrient targets comprise targets for at least protein, lipids, iron, calcium, and zinc.
